(19) United States
(12) Patent Application Publication
(10) Pub. No.: US 2024/0404621 A1
(43) Pub. Date: Dec. 5, 2024

(54) THERAPEUTIC DEVELOPMENT PLATFORM COMPRISING VARIANT CAPTURE MAPS AND OTHER VISUALIZATIONS

(71) Applicant: The Scripps Research Institute, La Jolla, CA (US)

(72) Inventors: Chao Wang, San Diego, CA (US); William E. Balch, San Diego, CA (US); Frédéric Anglès, La Jolla, CA (US)

(21) Appl. No.: 18/430,302

(22) Filed: Feb. 1, 2024

Related U.S. Application Data
(63) Continuation of application No. PCT/US22/39594, filed on Aug. 5, 2022.
(60) Provisional application No. 63/229,940, filed on Aug. 5, 2021.

(51) Int. Cl. G16B 15/30; G16B 15/20; G16B 45/00

(52) U.S. Cl. CPC G16B 15/30; G16B 15/20; G16B 45/00

(57) ABSTRACT

Drug discovery methodologies are enhanced through the use of variant capture maps that provide visualizations of functional covariance between different amino acids of a protein. These variant capture maps can be filtered with 3D distance data and overlapped to provide a rich source of information regarding sequence, structure, and function of a protein to assist development of treatment compounds and protocols.

### RELATED APPLICATIONS

This application is a continuation of PCT Application PCT/US2022/039594, which application claims priority to U.S. Provisional Application 63/229,940, filed on Aug. 5, 2021 and entitled Therapeutic Development Platform for Human Disease by Triangulating Genetic Variation in the Population. The entire disclosure of this priority application is hereby incorporated by reference in its entirety.

### BACKGROUND

The present disclosure relates to optimizing genetic disease diagnoses, pharmaceutical treatment strategies and drug discovery procedures. For many diseases or other phenotypes, amino acid sequence variants in one or more proteins have been associated with the condition, and in some cases the relationship between the variant and the disease is at least partially characterized. This evolutionary derived information can be used to evaluate and diagnose patients, develop treatment protocols and guide drug discovery.

However, given the unique complex genetic or environmental background of each individual, the phenotype and/or therapeutic response of a variant(s) in the population could be diverse. Moreover, for many variants, the available information is incomplete, and the relationship between the variant and a phenotype is unknown. With the lack of understanding of the mechanisms linking genotype to phenotype, developing a new drug to precisely manage human disease is often challenging. Currently, determining the high-resolution structure of a complex between the small molecule and the target protein is a favored approach to reveal the underlying molecular features of small-molecule therapeutics on the biology affecting the protein fold.

Recent advances in cryoelectron microscopy (cryo-EM) have enabled the detection and modeling of small-molecule interactions with different conformations of proteins and protein complexes. However, capturing key residue interactions in the protein fold design disrupted by genetic variants triggering human disease in the patient population and their responses to a therapeutic remains a challenge, as a disease-triggering variant frequently disrupts the intrinsic thermodynamic properties of the fold, hindering biochemical and biophysical characterization. This poses a major challenge for the development and application of therapeutics to manage disease not only in the population but the individual.

### SUMMARY

Understanding and predicting the impact of inherited and somatic mutation on human health is a central goal of precision medicine. Described herein is a technique referred to as Variation Capture (VarC) that is based on the new principle of ‘spatial covariance-based triangulation’ (SCVT). SCVT utilizes genetic variation in the population to deduce genotype to phenotype relationships in humans or other organisms for development of therapeutics.

In one embodiment, a method of estimating clinical, biological and/or chemical properties of protein variants comprises estimating one or more clinical, biological, and/or chemical properties in the presence and absence of one or more therapeutic and/or environmental conditions for amino acid residue variation at all or substantially all amino acids of the amino acid sequence of the protein and estimating pairwise covariance of the estimated clinical, biological, and/or chemical properties in the presence and absence of at least one therapeutic or environmental condition for all or substantially all amino acid pairs of the protein. The method further comprises estimating a 3D distance between all or substantially all amino acid pairs of the protein and generating a visualization comprising a combination of the estimated pairwise covariance and the estimated 3D distance. In some embodiments the 3D distance threshold is between 10 angstroms and 50 angstroms. In some embodiments, the 3D distance threshold is about 30 angstroms. Estimating a 3D distance may be based at least in part on an experimentally or computationally determined 3D protein structure.

In another embodiment, a method of estimating clinical, biological and/or chemical properties of protein variants comprises estimating one or more clinical, biological, and/or chemical properties in the presence and absence of a first therapeutic and/or environmental condition for amino acid residue variation at all or substantially all amino acids of the amino acid sequence of the protein and estimating one or more clinical, biological, and/or chemical properties in the presence and absence of a second therapeutic and/or environmental condition for amino acid residue variation at all or substantially all amino acids of the amino acid sequence of the protein. The method further includes estimating pairwise covariance of the estimated clinical, biological, and/or chemical properties in the presence and absence of the first therapeutic or environmental condition for all or substantially all amino acid pairs of the protein and estimating pairwise covariance of the estimated clinical, biological, and/or chemical properties in the presence and absence of the second therapeutic or environmental condition for all or substantially all amino acid pairs of the protein. The method further comprises generating a visualization comprising a combination of the estimated pairwise covariance of the clinical, biological, and/or chemical properties in the presence and absence of the first therapeutic or environmental condition for all or substantially all amino acid pairs of the protein and the estimated clinical, biological, and/or chemical properties in the presence and absence of the second therapeutic or environmental condition for all or substantially all amino acid pairs of the protein. In some embodiments the first therapeutic and/or environmental condition comprises exposure to a first chemical compound. In some embodiments the second therapeutic and/or environmental condition comprises exposure to a second chemical compound. In some embodiments the second therapeutic and/or environmental condition comprises an environmental condition. Such as a temperature shift from normal body temperature.

The visualization may comprise a two-dimensional matrix with amino acid position in the sequence on both the x-axis and the y-axis. In the visualization, the degree of covariance for a given pair of amino acid residues may be indicated by a color and/or a shade of a color.

### BRIEF DESCRIPTION OF THE DRAWINGS

A more complete appreciation of the subject matter of the present disclosure and of the various advantages thereof can be realized by reference to the following detailed description in which reference is made to the accompanying drawings in which:

FIG. 1 illustrates a base workflow for variant analysis in some embodiments.

FIG. 2 illustrates a process of variant spatial profiling applied to the CFTR protein, variants of which are associated with cystic fibrosis.

FIGS. 3A through 3C illustrate example VSP heat maps.

FIGS. 4A through 4C illustrate example Variant Capture (VarC) maps.

FIGS. 5A through 5C show annotated CFTR structures annotated with on-diagonal covariance information from the VarC maps of FIGS. 4A through 4C.

FIG. 6A illustrates a VarC map showing covariance of CFTR CICon in response to LUMA and a CFTR structure annotated with on-diagonal covariance information from the VarC map.

FIG. 6B illustrates a VarC map showing covariance of CFTR TrIdx in response to ELEXA and a CFTR structure annotated with on-diagonal covariance information from the VarC map.

FIG. 6C illustrates overlap between residues with VarC covariance response and residues associated with computationally predicted CFTR ligand binding pockets.

FIG. 6D illustrates LUMA bound to a first predicted CFTR ligand binding pocket.

FIG. 6E illustrates ELEXA bound to a second predicted CFTR ligand binding pocket.

FIG. 7 illustrates a first extended workflow for variant analysis in some embodiments.

FIG. 8A illustrates a VarC map modified to incorporate 3D structural distance information.

FIG. 8B is a CFTR structure annotated with off-diagonal covariance information from the modified VarC map of FIG. 8A.

FIGS. 9A through 9C illustrate additional examples of VarC maps modified to incorporate 3D structural distance information.

FIGS. 9D through 9F illustrate binning of residue pairs based on strength of covariance.

FIGS. 9G through 9I illustrate CFTR structures annotated with off-diagonal covariance information from the modified VarC maps of FIGS. 9A through 9C.

FIG. 10 illustrates a second extended workflow for variant analysis in some embodiments.

FIGS. 11A and 11B illustrate overlapped modified VarC maps and CFTR structures annotated with off-diagonal covariance information from the overlapped modified VarC maps.

FIG. 12A illustrates a VarC map showing covariance of CICon in response to a temperature shift.

FIG. 12B illustrates a CFTR structure annotated with off-diagonal covariance information from the VarC map of FIG. 12A.

FIG. 12C illustrates a VarC map showing covariance of CICon in response to LUMA/IVA combination therapeutic treatment.

FIG. 12D illustrates a CFTR structure annotated with off-diagonal covariance information from the VarC map of FIG. 12C.

FIG. 12E illustrates overlapped VarC maps from FIG. 12A and FIG. 12C modified to incorporate 3D structural distance information.

FIG. 12F illustrates a CFTR structure annotated with off-diagonal covariance information from the VarC map of FIG. 12E.

FIG. 13 illustrates VarC maps and annotated CFTR structures related to CFTR TrIdx response to temperature shift.

FIG. 14 illustrates VarC maps and annotated CFTR structures related to CFTR TrIdx response to ELEXA and TRIKA therapeutic treatments.

FIG. 15 illustrates VarC maps and annotated CFTR structures related to CFTR CICon in response to ELEXA and TRIKA therapeutic treatments.

FIG. 16 illustrates details of covariance response for residues associated with the YKDAD region of the CFTR sequence.

FIG. 17 illustrates CFTR ligand binding pockets identified by the VarC mapping analysis described herein suitable for targeted drug discovery.

### DETAILED DESCRIPTION

Variation is foundational for driving evolvability and diversity in biology. It reports on precision information at the genome level required for biology to communicate with the environment to drive development and achieve survival and fitness through natural selection. Individual susceptibility and clinical presentation for human disease is strongly impacted by genetic variation. Understanding how genetic variation shapes the functional protein fold in the context of environment is crucial to guide an understanding of the variant complexity in the genome and proteome that makes each one of us unique across the population and to inform precision therapeutic management of human disease. To address quantitatively the basis for variation in human health and disease the inventors have recently shown that genetic diversity distributed across the genomes of the world-wide population can be rigorously framed through variation spatial profiling (VSP) to achieve a deeper understanding of sequence-to-function-to-structure relationships. VSP may in some embodiments build phenotype landscapes based on the principle of spatial covariance (SCV) in biological systems to establish predictions regarding the function of each amino acid residue across an entire polypeptide/protein sequence using only a sparse collection of variants found in the population. SCV defines the role of each amino acid residue encoded in a gene by linking its linear sequence position to its multi-dimensional functional roles in biology in the context of other residues to amplify our understanding of the polypeptide chain at atomic resolution. Described herein is a method to capture SCV relationships in a combined pairwise-residue framework by triangulating a sparse collection of genome variation to understand dynamic protein function-structure relationships in response to changing environments. This “SCVT” approach may utilize weighted proximity in a GPR-ML based matrix to define precisely the functional defects driving a disease phenotype that provides a novel basis for assigning the atomic trajectories for the discovery of small molecules that alone or as combination can correct functional defects in the fold contributed by aberrations in protein folding in response to genetic disease.

To understand how the genotype to phenotype transformation is shaped by the environment to inform disease management, we developed variation-capture (VarC) mapping. VarC triangulates sparse sequence variation information across the population using a unique application of Gaussian process regression (GPR) based machine learning to capture combined pairwise-residue interactions contributing to dynamic proteome function in the individual in response to physical, chemical and molecular environment. VarC mapping generates a quantitative approach based on the evolutionary roots of genetic disease distributed in the population to define the functional trajectory of the protein fold that drives human biology in the individual. VarC provides a universal tool utilizing the spatial covariant design of information flow linking genome variation to proteome function to inform precision management of human health and disease in the individual at functionally defined atomic resolution. Herein, we develop a Variation Capture (VarC) strategy that triangulates the sparse sequence information in the population to define the combined pairwise-residue basis the information flow from the genome to the proteome to link genetic variation to the function of protein fold in response to physical, chemical and molecular environments.

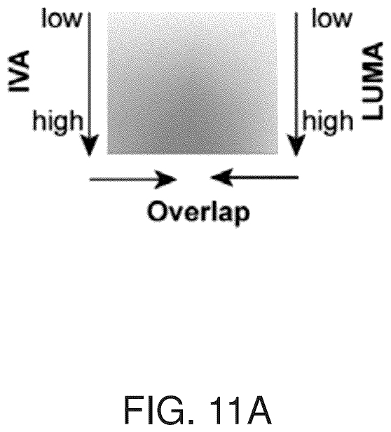

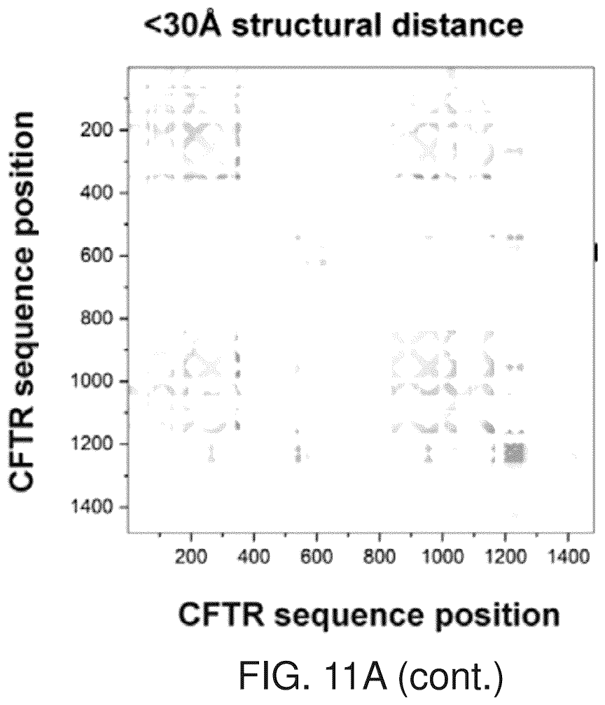

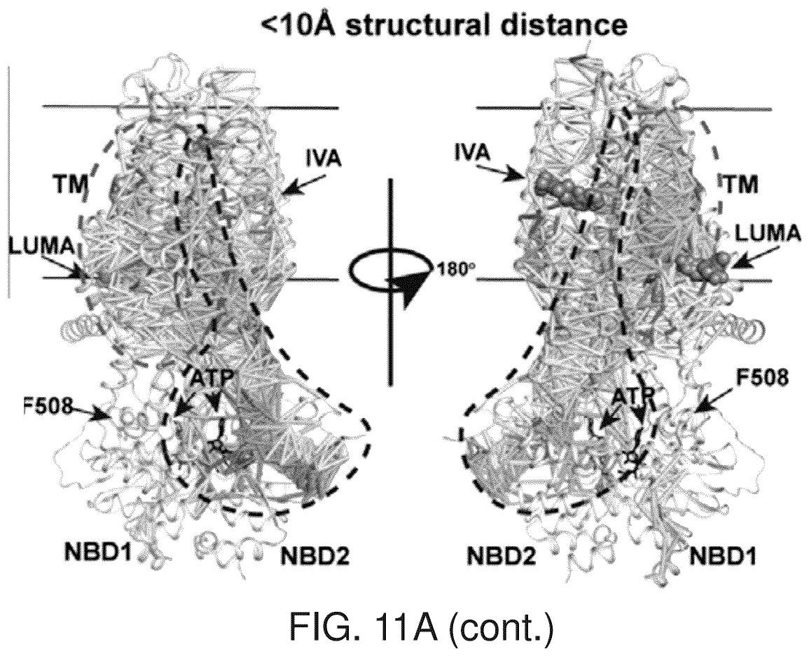

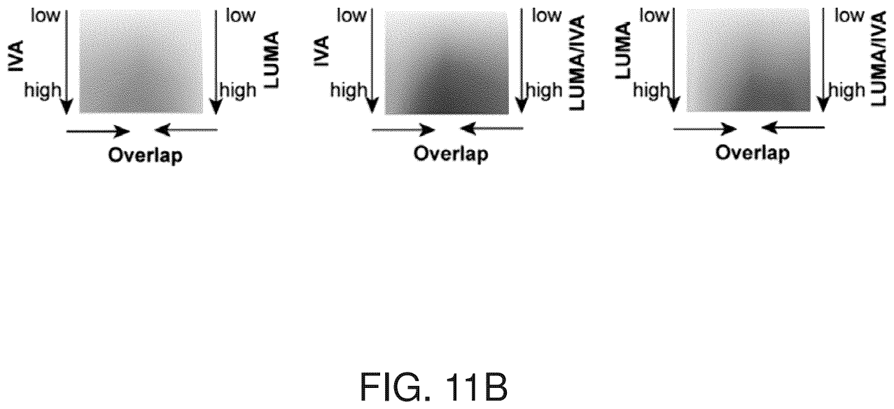

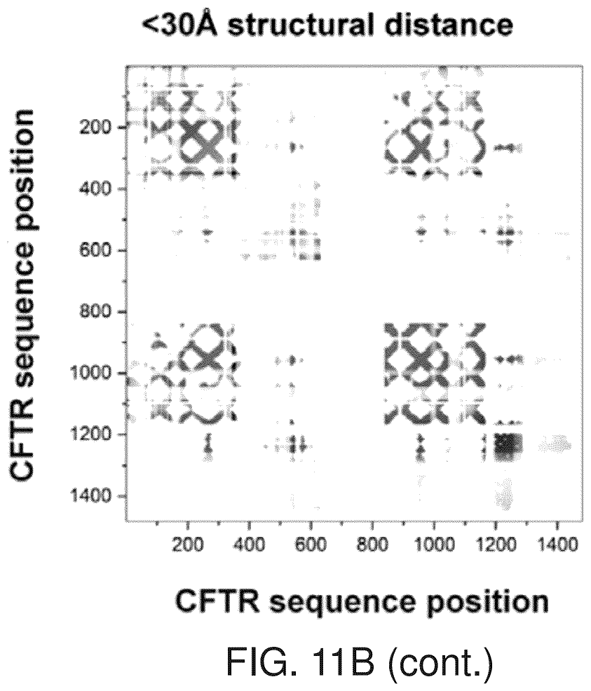

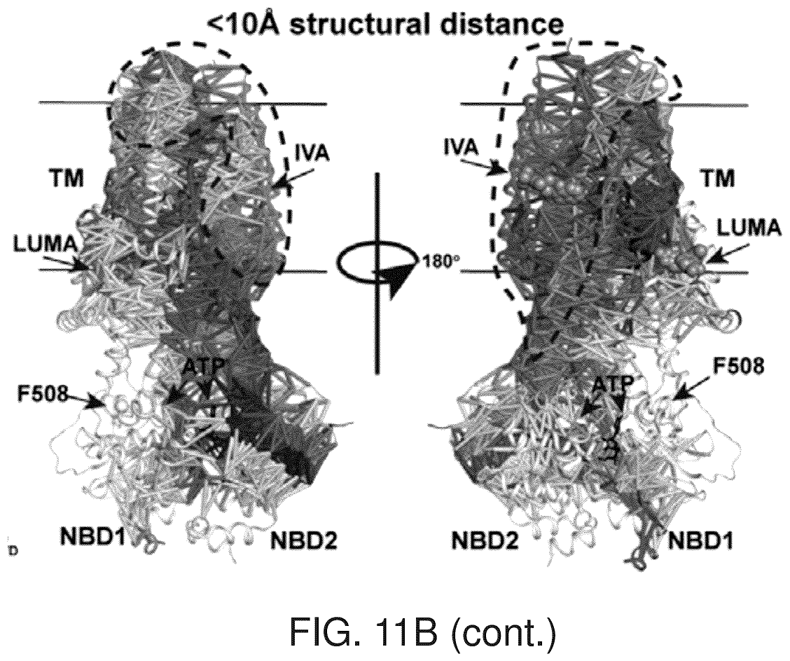

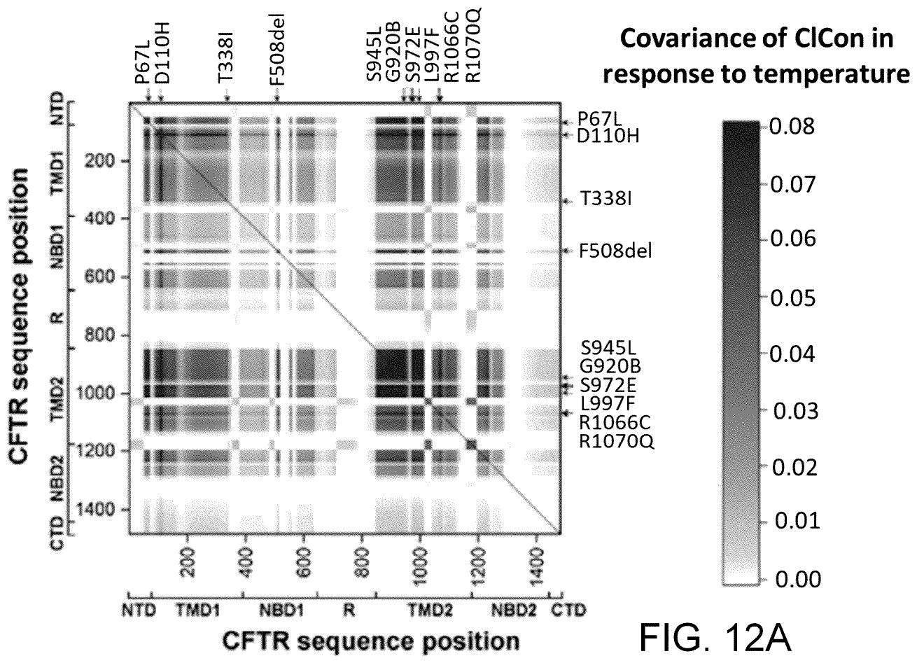

SCV based VarC mapping provides a rigorous and quantitative GPR-ML based approach to capture information flow from the genome to the proteome to understand function in protein fold design. VarC mapping now suggests that a major weakness in understanding the impact of any variant (inherited or somatic) on human health is not necessarily the variant itself, but the variant in the context of altered SCV based relationships combined pairwise-residue relationships (a biological ‘black hole’ (FIG. 11B)). We posit that the level of mechanistic understanding gained by using only a sparse collection of variants in the population in the context VarC mapping that captures diversity in the population illustrates the potential of the VarC analysis as a generalized platform to more fully characterize the role of evolving biological pathways responsive to the environment to define physiological function of the fold. This conclusion is consistent with the fact that SCV relationships encoded in the sequence of the genome tells the proteome how to fold for function; the proteome through SCV in response to the environment tells the genome how to shape biology. Using the VarC approach to triangulate the multi-dimensional features contributing to the spatial covariant design of the information flow linking genome variation to proteome function through central dogma in the population will inform atomic resolution mapping of the functional SCV trajectories for precision management of health and disease in the individual. By illuminating the impact of a therapeutic using a SCV-principled approach based on combined pairwise-residue covariance relationships impacting the functional structure, VarC provides a tool to generate a therapeutic solution to the folding problem(s) compromised in human genetic disease.

To illustrate how this general framework can be used to universally address the role of pairwise-residue relationships driving sequence-to-function-to-structure design of the protein fold in response to environment, the role of genetic variation in cystic fibrosis (CF) is discussed herein. CF is one of the most common rare diseases, impacting ˜70,000 individuals worldwide with over 2000 variants in the cystic fibrosis transmembrane conductance regulator (CFTR). CFTR is a key chloride conductance (CICon) channel that traffics from the endoplasmic reticulum (ER) to the apical cell surface to maintain critical ion balance in sweat, intestinal, pancreatic, and pulmonary tissues. Each variant in CFTR contributes to the protein fold in unique ways in the individual presenting a challenge to manage in the clinic. CF has been the poster child for drug development with the recent advent of the Trikafta (TRIKA). TRIKA is a combination mix containing Tezacaftor (TEZA) (an analogue of Lumacaftor (LUMA)), a potential corrector of ER export deficiencies, and Ivacaftor (IVA), a potentiator of gated channel function, augmented with Elexacaftor (ELEXA), a purported corrector. TRIKA achieves a substantial improvement of basic and clinical features of disease. Their roles as chemically based modulator therapies, like most therapeutics, in the context of the genotype to phenotype transformation across the polypeptide chain, are largely unknown.

The variants of triangulation technology described in this disclosure not only provide a platform to screen drugs with new correction mechanism for cystic fibrosis, but also can be generalized to any genetic disease to facilitate the design of experimental HTS, HCIS or in silico screening for therapeutic development. A GPR-ML based covariance platform for any protein defined by variation in the population can provide a universal tool to reveal the evolutionary trajectories in the protein fold at atomic resolution for precision management of human health and disease in the individual.

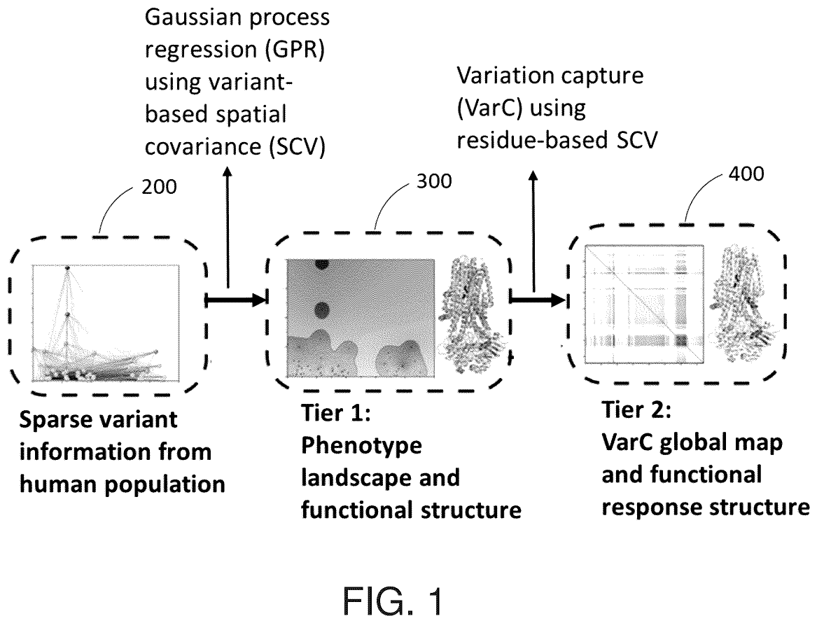

### Base Workflow Overview—FIG. 1

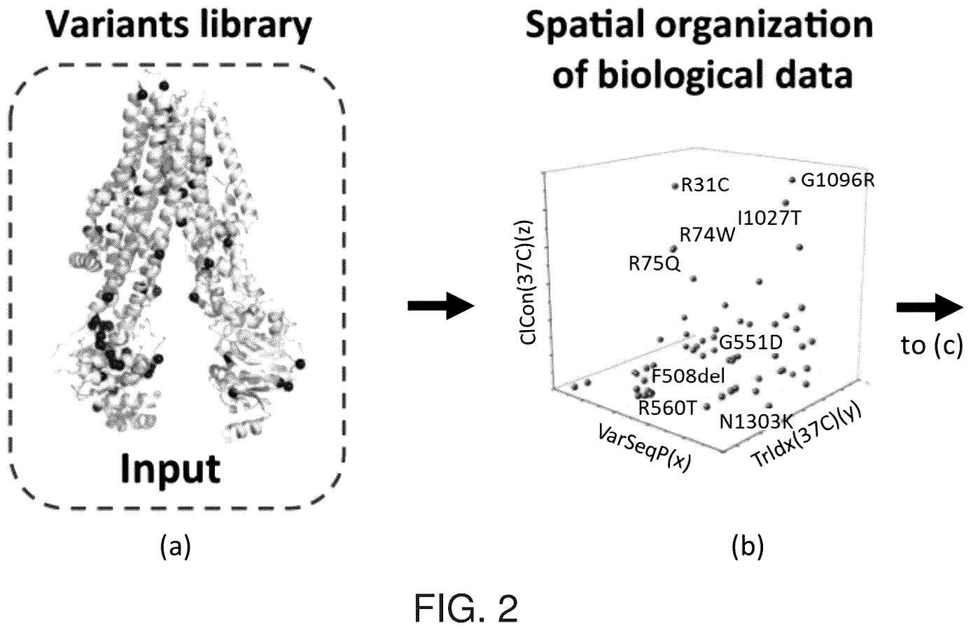

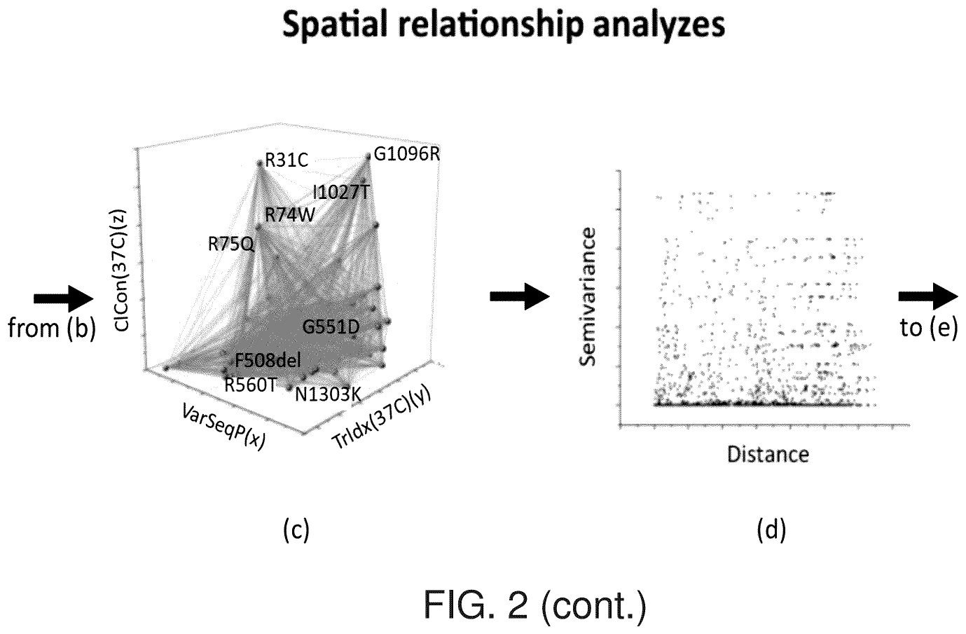

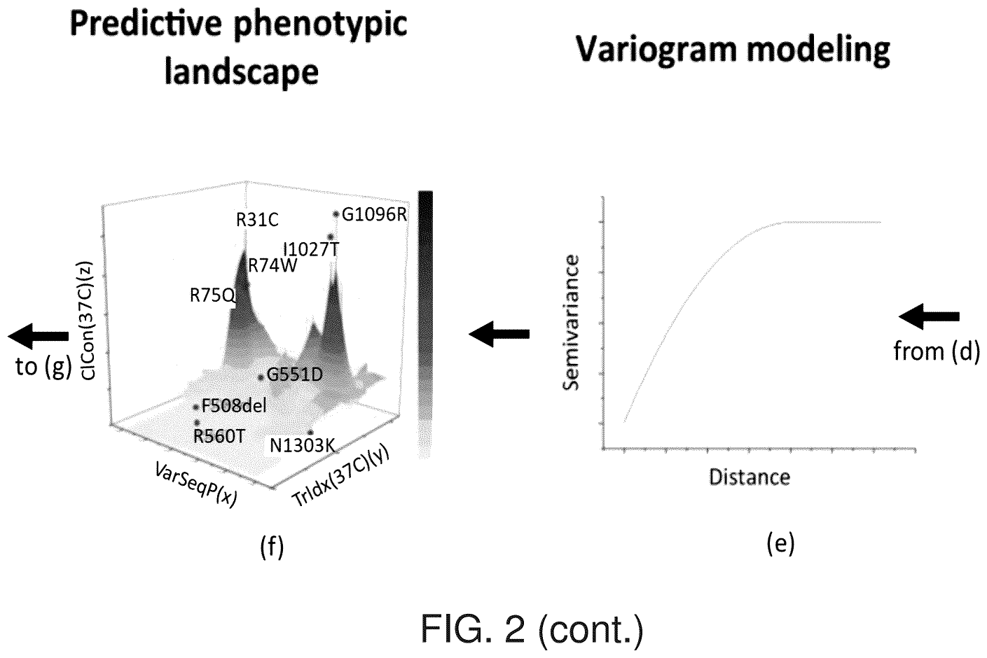

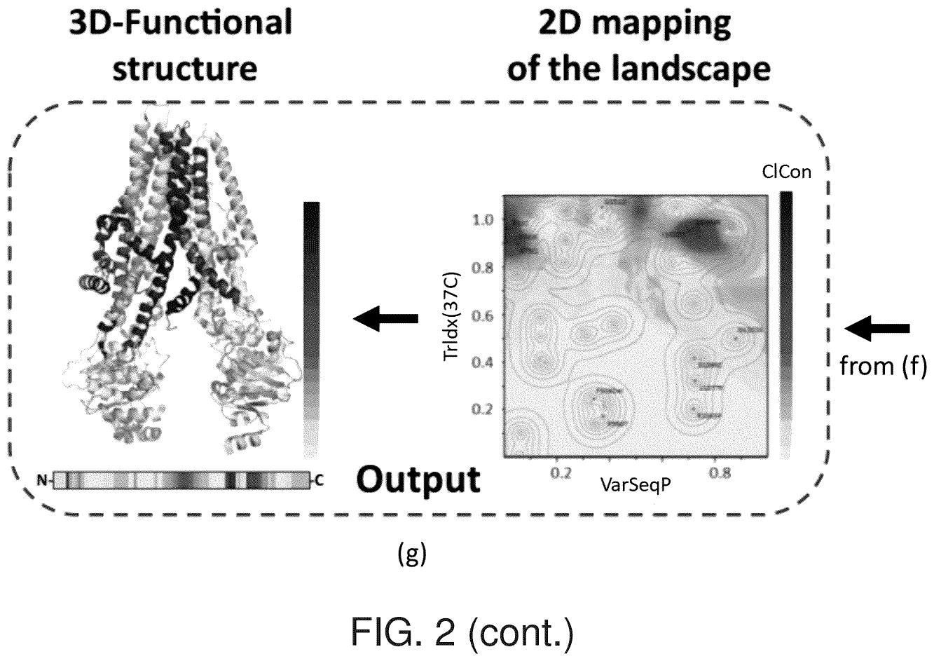

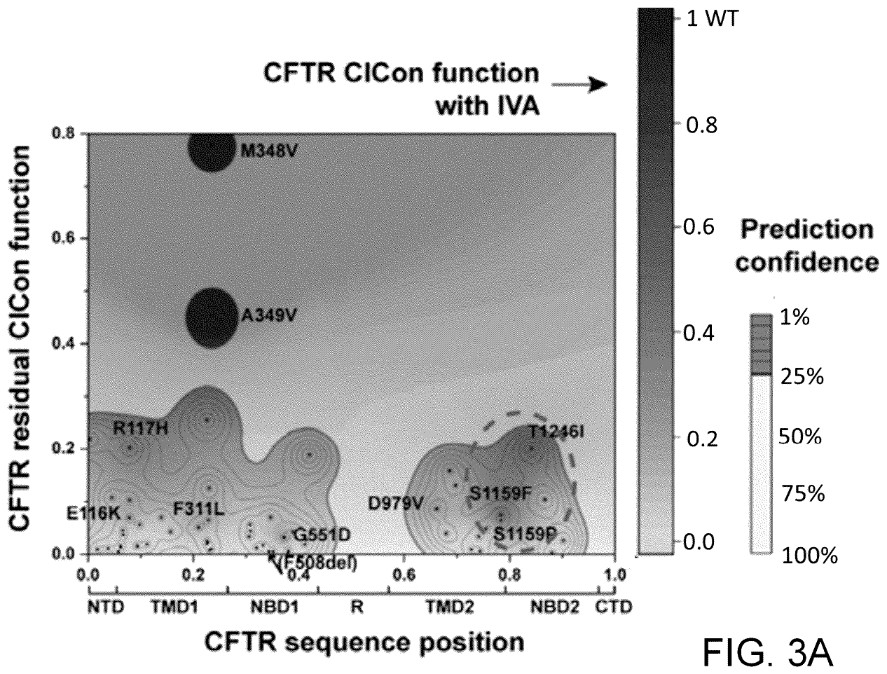

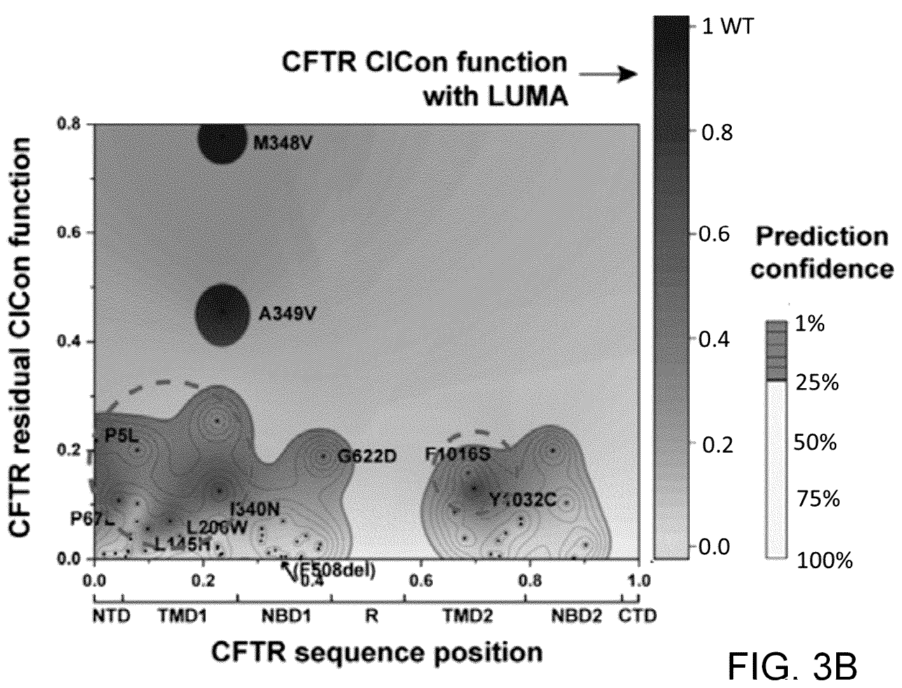

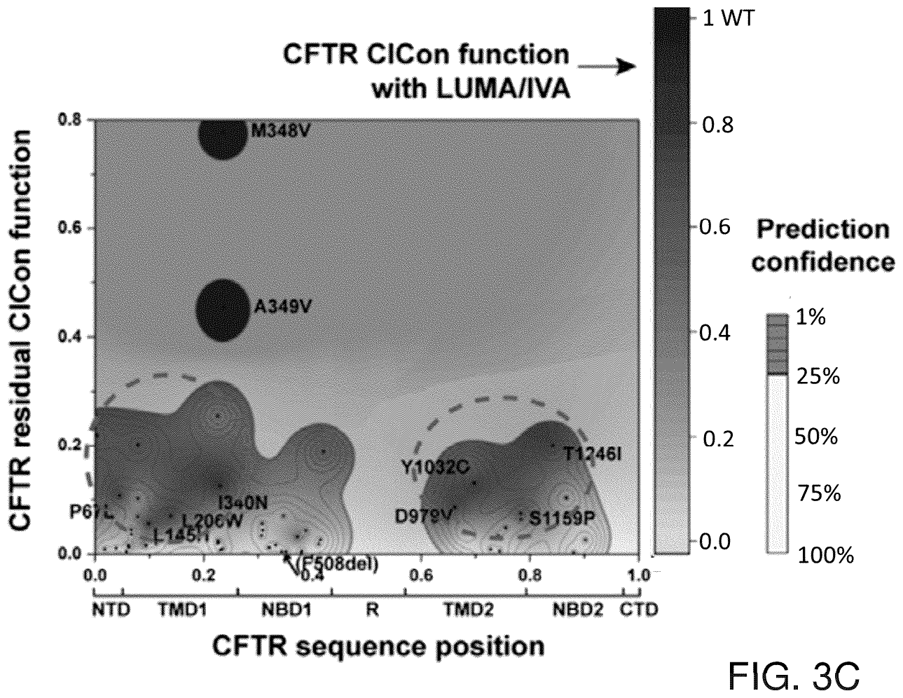

Referring now to FIG. 1, at block 200 the “base workflow” method may have as an input a sparse set of variant information related to variant location in an amino acid sequence and information regarding one or more chemical, biological, or clinical characteristics of each input variant. At 300, the variant based SCV relationships may be assessed using VSP where a phenotype landscape and its corresponding functional-structure are built to predict the function of all residues based on the information from a sparse collection of input variants. Blocks 200 and 300 are described in more detail with reference to FIGS. 2 and 3 below.

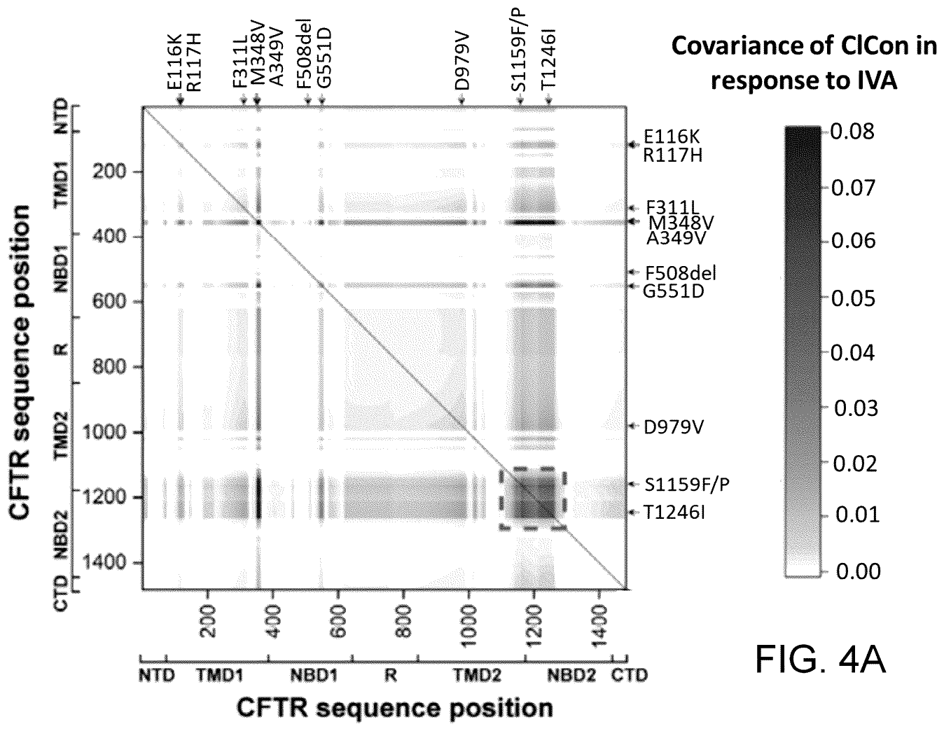

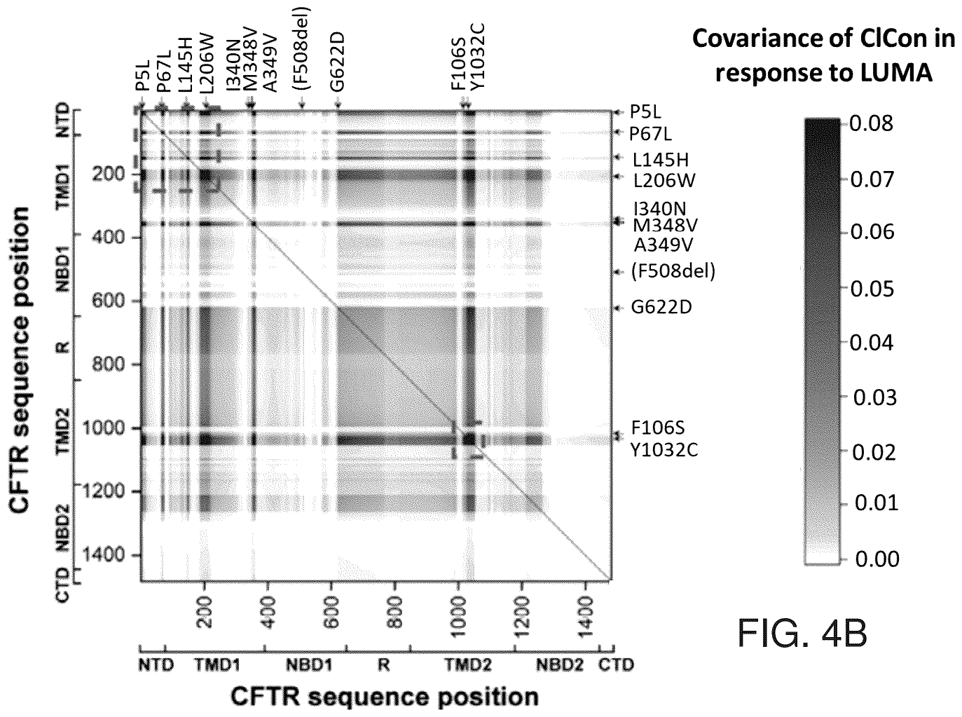

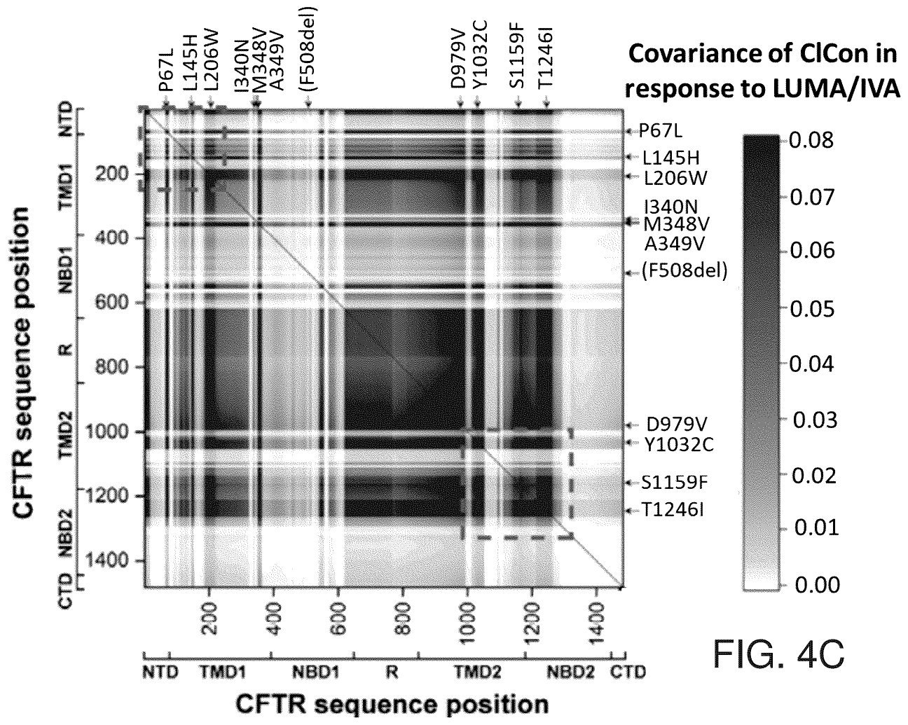

At 400, the analysis computes pairwise-residue based SCV relationships on the functional response to a physical or chemical treatment to construct a “Variation Capture” (VarC) map that defines how each residue covaries with every other residue across the entire sequence to drive the functional response. In the VarC map at 400, the on-diagonal values report the residue-by-residue response to a treatment as the variance value (the square of the standard deviation) between the residual (no treatment) and the treated response for each residue. The off-diagonal values in VarC maps present the residue-to-residue functional covariance in response to the treatment for any two different residues. On-diagonal values may be used to build a ‘functional-response-structure’ to define the individual role of each residue in the polypeptide sequence function in response to a treatment. The VarC map at 400 is described in more detail below with reference to FIG. 4. Examples 1 and 2 below utilize the information and visualization methods illustrated in block 400. The VarC maps and other graphic displays (e.g. annotated 3D protein structures) described herein are visualizations useful for drug discovery that can be presented to users on user interfaces such as computer displays.

FIG. 2 illustrates an example process of variant spatial profiling (VSP) to generate a phenotype landscape 300 of FIG. 1. VSP uses a sparse collection of variants with known position and phenotypical traits. Each of the variants is positioned on the x-axis by their normalized position in the linear polypeptide sequence, where the full-length chain is assigned a value of 1.0, referred to as the variant sequence position (VarSeqP). The value of a phenotypic feature of each variant's (in this example the trafficking index, TrIdx) to the y-axis. The trafficking index (TrIdx) is the normalized ratio of the fraction of variants exported from the ER relative to the total variant level in the cell, where the value of the WT CFTR TrIdx is assigned a value of 1.0. In this example, the sequence position and TrIdx values are correlated to CFTR chloride conductance (CICon). The normalized CICon is assigned to the z-coordinate, where the value of the WT CFTR CICon is assigned a value of 1.0.

The 2D distance values based on VarSeqP and TrIdx, as well as the variance of CICon for all possible 2080 (based on 64 variants) variant pairwise combinations may be computed. A molecular variogram shows the calculated spatial variance and distance values for each comparison. Such modeling quantitatively defines the linear sequence range where the variants co-vary with each other for a given set of functions defined by the y- and z-axis coordinates (TrIdx and CICon, respectively) until it reaches a plateau that may be referred to as the ‘molecular range’. Variants with distance relationships extending beyond the range are generally not correlated. The molecular variogram is used to predict all uncharacterized impacts of amino acid residue variation on the CICon (z-axis) based on the residue position in the polypeptide sequence (x-axis) and the TrIdx (y-axis).

For the variogram analysis, the measured variants may be positioned by their sequence positions in the polypeptide chain on the ‘x’ axis coordinate and their measured residual feature (e.g., TrIdx or CICon function) without treatment on the ‘y’ axis coordinate to the measured feature with treatment (e.g., compounds or low temperature) along the ‘z’ axis coordinate. Suppose the i^{th} (or j^{th}) observation in a dataset consists of a value zi (or zj) at coordinates xi (or xj) and yi (or yj). The distance h between the i^{th} and j^{th} observation is calculated by:

$${h}_{( i , j )} = \sqrt{{( {x}_{i} - {x}_{j} )}^{2} + {( {y}_{i} - {y}_{j} )}^{2}} \qquad ( 1 )$$

The γ(h)-variance for a given distance (h) is defined by:

$$γ ( h ) = \frac{1}{2} {( {z}_{i} - {z}_{j} )}^{2} \qquad ( 2 )$$

where (h)-variance is the semivariance (i.e., the degree of dissimilarity) of the z value between the two observations, which is also the whole variance of z value for one observation at the given separation distance h, referred as spatial variance here. The distance (h) and spatial variance (γ(h)) for all the data pairs are generated by the equations (1) and (2). Then, the average values of spatial variance for each distance intervals (0.01 used in this study) are calculated to plot the averaged spatial variance versus distance. Spherical model is used to fit the variogram in this study. The molecular variogram defines quantitatively the correlation between the spatial variance of z changes and the separation distance defined by the x and y coordinates based on known variants. The distance where the model first flattens out is known as the range. Locations separated by distances closer than the range are spatially correlated, whereas locations farther apart than the range are not. The variogram enables us to compute the spatial covariance (SCV) matrices for any possible separation vector. The SCV at the distance (h) is calculated by C(h)=C(0)−γ(h), where C(0) is the covariance at zero distance representing the global variance of the data points under consideration (i.e., the plateau of the variogram).

VSP aims to generate the prediction that has minimized estimation error, i.e., error variance, which is generated according to the expression:

$${σ}_{u}^{2} = E [ {( {Z}_{u}^{*} - {Z}_{u} )}^{2} ] = {\sum }_{i = 1}^{n} {\sum }_{j = 1}^{n} {ω}_{i} {ω}_{j} {C}_{i , j} - 2 {\sum }_{i = 1}^{n} {ω}_{i} {C}_{i , u} + {C}_{u , u} \qquad ( 3 )$$

where zᵤ* is the prediction value while zᵤ is the true but unknown value, C_{i,j} and C_{i,u} are SCV between data points i and j, and data points i and u, respectively, and C_{u,u} is the SCV within location u. ωi is the weight for data point i. The SCV is obtained from the above molecular variogram analysis and the weight (ωᵢ) solved from equation (3) is used for following prediction. To ensure an unbiased result, the sum of the weights is set as one:

$${\sum }_{i = 1}^{n} {ω}_{i} = 1 \qquad ( 4 )$$

Equations (3) and (4) not only solve the set of weights associated with input observations, but also provide the minimized ‘molecular variance’ at location u which can be expressed as:

$${σ}_{u}^{2} = {C}_{u , u} - ( {\sum }_{i = 1}^{n} {ω}_{i} {C}_{i , u} + μ ) \qquad ( 5 )$$

where Cu,u is the SCV within location u, ωi is the weight for data point i, and Ci,u are SCV between data points i and u. μ is the Lagrange Parameter that is used to convert the constrained minimization problem in Eq. (3) into an unconstrained one. The resulting minimized molecular variance assessing the prediction uncertainty presents the confidence level of the prediction.

The minimization of variance (equation 3) with the constraint that the sum of the weights is 1 (equation 4) can be written in matrix form as:

$$C \cdot W = D \qquad ( 6 )$$

$$[ \begin{matrix}{C}_{1 , 1} & … & {C}_{1 , n} & 1 \\ ⋮ & ⋱ & ⋮ & ⋮ \\ {C}_{n , 1} & … & {C}_{n , n} & 1 \\ 1 & … & 1 & 0\end{matrix} ] \cdot [ \begin{matrix}{ω}_{1} \\ ⋮ \\ {ω}_{n} \\ μ\end{matrix} ] = [ \begin{matrix}{C}_{1 , u} \\ ⋮ \\ {C}_{n , u} \\ 1\end{matrix} ]$$

where ‘C’ is the covariance matrix of the known data points. ‘W’ is the set of weights assigned to the known data points for generating the predicted phenotype landscape. ‘u’ is the Lagrange multiplier to convert a constrained minimization problem into an unconstrained one. ‘D’ is the covariance matrix between known data points to the unknown data points. Since ‘W’ is the value we want to solve to generate the phenotype transformation (the phenotype landscape), this equation can be also written as:

$$W = {\underbrace{{C}^{- 1}}}_{Clustering} \cdot {\underbrace{D}}_{Distance} \qquad ( 7 )$$

where ‘C⁻¹’ is the inverse form of the ‘C’ matrix. As a more intuitive explanation of the matrix notation, herein we simply refer to the VSP matrix that generates the phenotype landscape (‘W’) to be based on the two important computational features used for predicting the unknown function values from the known—(1) the clustering (i.e., clustered sequence values with similar functional properties ‘C⁻¹’) and (2) the distance constraints (D). Here, ‘C⁻¹’ represents the clustering information of the known data points while ‘D’ represents predicted statistical distance between known data points to unknown data points.

With the solved weights W, we can calculate the prediction of all unknown values to generate the complete phenotype landscape by the equation:

$${z}_{u}^{*} = \text{?} {ω}_{i} {z}_{i} \qquad ( 8 )$$

$$\text{?} \text{indicates text missing or illegible when filed}$$

where zᵤ′ is the prediction value for the unknown data point u, ωᵢ is the weight for the known data point, and zᵢ is the measured value for data point i.

The 3D phenotype landscape may be projected as a 2D heatmap of the z-coordinate CICon values to generate a 2D phenotype landscape. A confidence interval (uncertainty) can be assigned to every data point that can be delineated by contour lines in the 2D projection map. The generated CICon-phenotype landscape values with the highest confidence for all CFTR residues may be projected on a 3D protein structure model to create a CICon functional structure displaying how amino acid variation at each position is impacting the functional-structure relationships.

This procedure is further described in Wang, C. & Balch, W. E. “Bridging genomics to phenomics at atomic resolution through variation spatial profiling.” Cell Rep. 24, 2013-2028 (2018); Anglès, F., Wang, C. & Balch, W. E., “Spatial covariance analysis reveals the residue-by-residue thermodynamic contribution of variation to the CFTR fold,” Commun Biol 5, 356 (2022); and US Patent Application Publication 2021/0324474, entitled “Methods for Disease Treatment and Drug Discovery,” all three of which are incorporated by reference herein in their entireties.

FIGS. 3A, 3B, and 3C show example heat maps corresponding to block 300 of FIG. 1 where the first phenotype characteristic (y-axis) is a variant function measurement (CICon in the FIG. 3A through 3C examples) and the second phenotype characteristic (z-axis or color) is a response of the first phenotype characteristic to a particular therapeutic treatment. The example of FIG. 3A illustrates predicted CICon response to Ivacaftor (IVA) treatment at every residue of the CFTR protein, the example of FIG. 3B illustrates predicted CICon response to Lumacaftor (LUMA) treatment at every residue of the CFTR protein, and the example of FIG. 3C illustrates predicted CICon response to a combination LUMA/IVA treatment at every residue of the CFTR protein.

FIGS. 4A, 4B, and 4C show three example VarC maps based on the three phenotype landscape maps shown in FIGS. 3A, 3B, and 3C. These correspond to block 400 of the base method of FIG. 1. The VarC map is a covariance matrix that captures both the local and long-range association of any pairwise-residues along the primary sequence in response to a treatment based on the prediction from phenotype landscape. Phenotype landscapes generated through VSP predict the full range of values describing the function under treatment (i.e., z-axis value) with associated residual function without treatment (i.e., y-axis value) for the entire polypeptide sequence (x-axis). To build the VarC map, the prediction value with the lowest estimate variance (i.e., highest confidence) for each residue may be used. For any two residues A and B, there is the most confident prediction under the treatment condition (At or Bt) and the associated value without treatment (A0 or B0). Then, the covariance matrix of any pair of residues for the entire polypeptide is calculated by:

$$Cov ( A , B ) = ( {A}_{0} - \overline{A} ) ( {B}_{0} - \overline{B} ) + ( {A}_{t} - \overline{A} ) ( {B}_{t} - \overline{B} )$$

$$where$$

$$\overline{A} or \overline{B}$$

is the average between untreated value and the compound treated value (or low temperature treatment where applicable). The diagonal value of the VarC map is the covariance with the residue itself, i.e., the variance between the untreated value and the value under treatment. For example:

$$Cov ( A , B ) = {( {A}_{0} - \overline{A} )}^{2} + {( {A}_{t} - \overline{A} )}^{2}$$

$$where$$

$$\overline{A}$$

is the average between untreated value and the value under compounds or low temperature treatment. To generate the functional-response-structure (right side of block 400), the diagonal values of the VarC map may be projected to each residue of the polypeptide or protein of interest, such as on the cryo-EM structure of CFTR (PDB:5UAK for closed conformation, PDB:6MSM for open conformation and 6O2P for Ivacaftor binding complex) with corresponding color scale assigned using PyMol software.

As noted above, in a VarC map, the on-diagonal values report the residue-by-residue response to a treatment as the variance value (the square of the standard deviation) between the residual (no treatment) and the treated response for each residue. The off-diagonal values in VarC maps present the residue-to-residue functional covariance in response to the treatment for any two different residues.

As will be discussed further below, the VarC map, visualizations derivable from the VarC map, and correlations between the VarC map content and other forms of structural and biological information form powerful tools to guide drug discovery from high-throughput in-silico compound screening to clinical trial design.

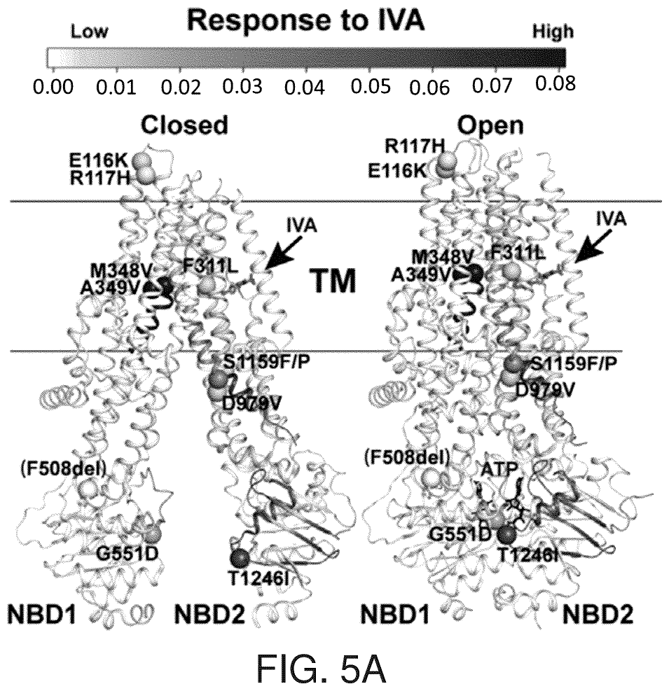

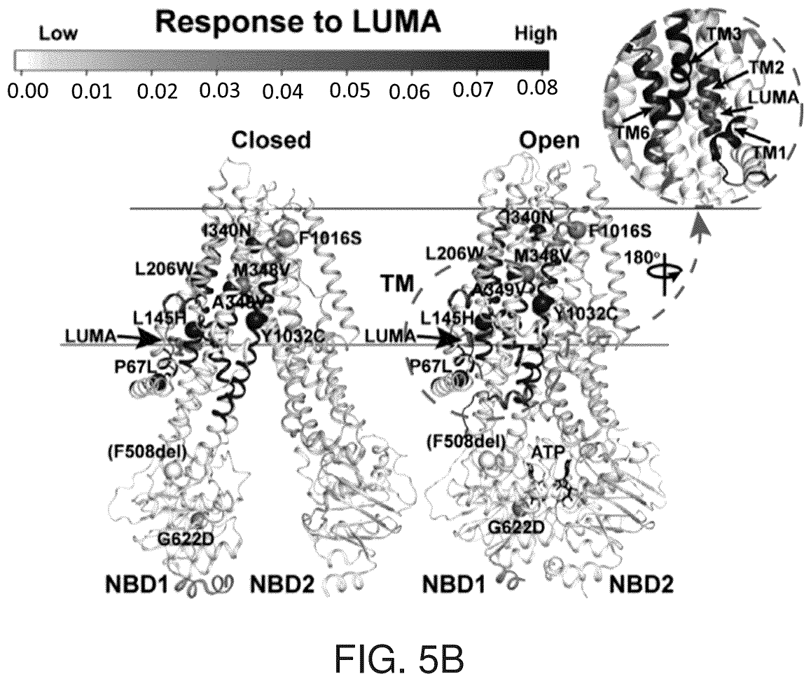

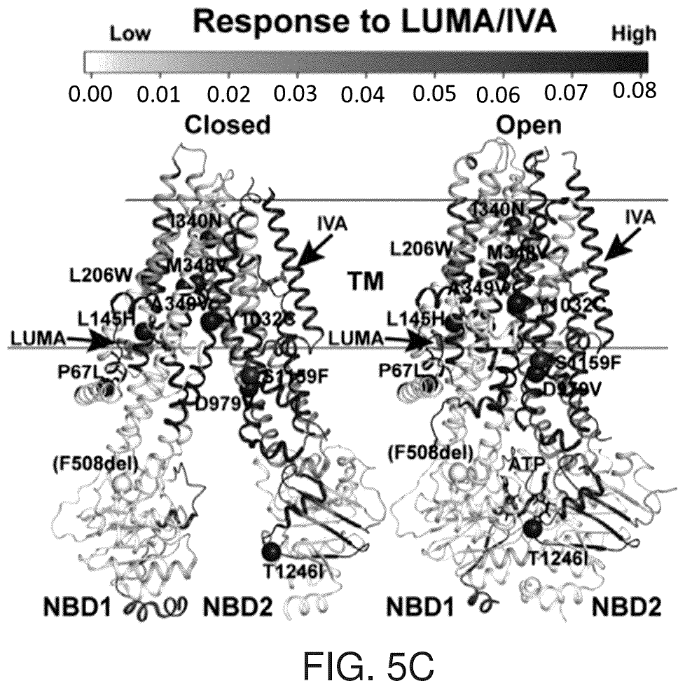

### Base Workflow Example 1—FIGS. 4-5

A better understanding of how different chemical modulators shape the polypeptide sequence-to-function relationships on a pairwise-residue basis across the entire polypeptide sequence, can be gleaned from the variant-based phenotype landscape predictions (FIG. 1, block 300, FIGS. 3A through 3C) to generate pairwise-residue based SCV relationships using variation-capture (VarC) mapping (FIG. 1, block 400, FIGS. 4A through 4C). VarC mapping may use the prediction with lowest uncertainty computed for every residue in the phenotype landscape to build a covariance matrix for all possible pairwise-residue associations along the polypeptide sequence in response to IVA (FIG. 4A), LUMA (FIG. 4B) and the LUMA/IVA mix (FIG. 4C). Each pairwise-residue based VarC plot reports 2,190,400 SCV defined functional covariance relationships encoded in the CFTR genome in response to different chemical environments. The values along the diagonal (the residue-by-residue relationships) of VarC maps present the variance value (the square of the standard deviation) between the residual (no modulator) and modulator treated CICon response. The off-diagonal values (the residue-to-residue relationships) in VarC maps present the functional covariance for any two different residues in CFTR, in this case in response to CF modulator(s) IVA and LUMA. As expected, the pairwise-residue matrices of CFTR response to IVA (FIG. 4A, z-axis color) and LUMA (FIG. 4B, z-axis color) are impacted differently by the two modulators to drive CICon correction. For example, many functional covariance signals responding to LUMA are clustered in the N-terminal of CFTR polypeptide (FIG. 5A, NTD-TMD1 domains) that are absent in the VarC plot for IVA (FIG. 5B). Furthermore, most pairwise responses are stronger in LUMA/IVA-VarC map (FIG. 4C) when compared to IVA-VarC map (FIG. 4A) or the LUMA-VarC map (FIG. 4B) reflecting potential additive or synergistic interactions.

To interpret the VarC map from a structural perspective to address how the linear polypeptide chain organizes 3D structure to implement the residue-based SCV relationships that manage information flow from genome variation to proteome function, we mapped the on-diagonal value of each residue in the VarC map to cryo-EM structures of CFTR in both closed and open conformations to generate ‘functional-response-structures’ (FIGS. 5A, 5B, 5C). While the strong responding residues for IVA are spread across different domains in the primary sequence (FIG. 5A), they are largely aligned along the CI channel in CFTR, consistent with the role of IVA for allosterically regulating the channel gating. Importantly, most of the predicted responding residues involve the transmembrane (TM) alpha helixes that connect to NBD2, consistent with the proposed IVA binding site as revealed in the cryo-EM structure.

In striking contrast, the high responsive residues for LUMA are clustered in the TM region projection (FIG. 5B) that contains distinct linear sequence regions in NTD, TMD1 and TMD2 domains. The predicted high responsive regions involving four α-helices (TM1-3 and TM6) in TMD1 were recently shown to form the binding pocket for LUMA in the cryo-EM structure of CFTR. This result strongly validates the residue-by-residue responses predicted by VarC to capture the binding mechanism of LUMA. Furthermore, VarC now reveals that the strong response of this cluster to LUMA is allosterically accompanied by many weak responding residues including those TM regions found linking the NBD2 domain to the C-terminal region of the NBD1 domain. The mix of LUMA and IVA yields a broader and higher CICon response across many residues (FIG. 5C), particularly for the regions around IVA binding site (FIG. 51, IVA arrow), which suggests a synergistic effect between LUMA and IVA modulators to potentiate the CFTR channel function, revealing the potential of VarC to capture a wide range of dynamic interactions in protein folds.

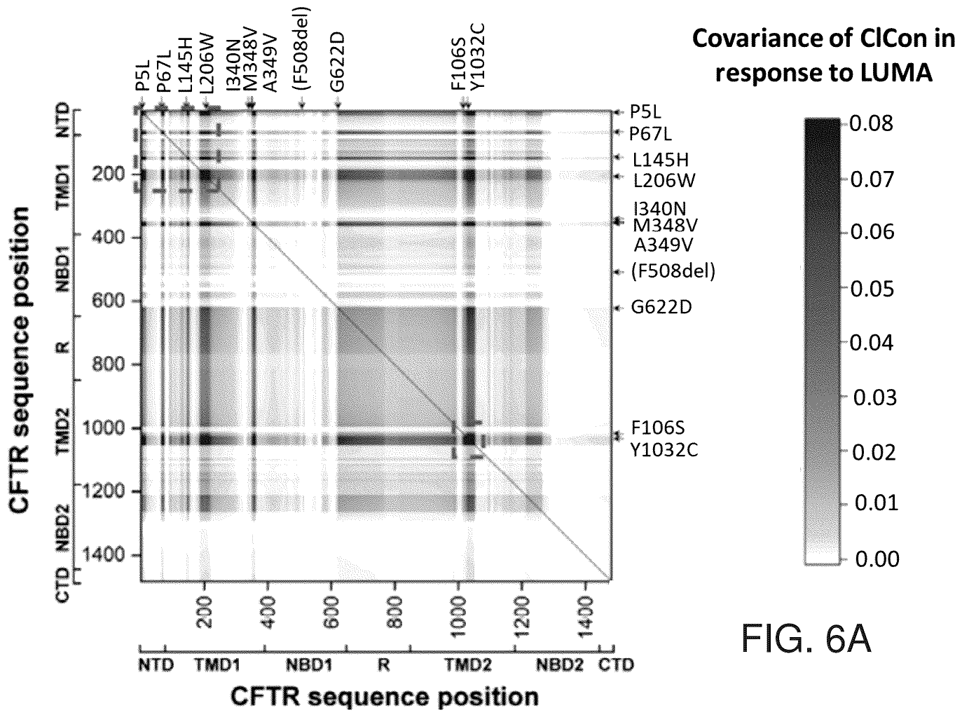

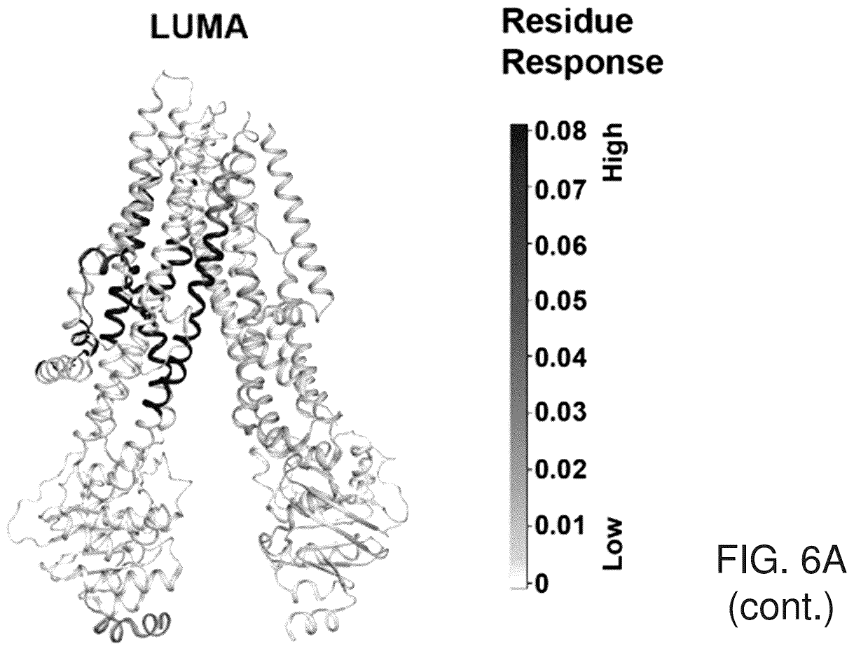

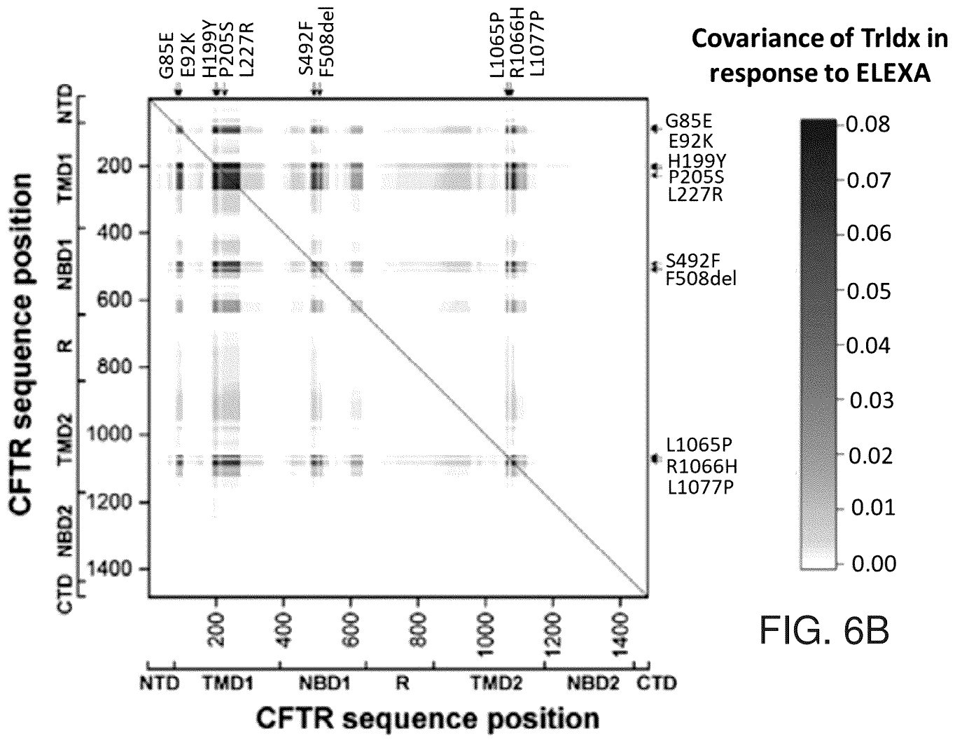

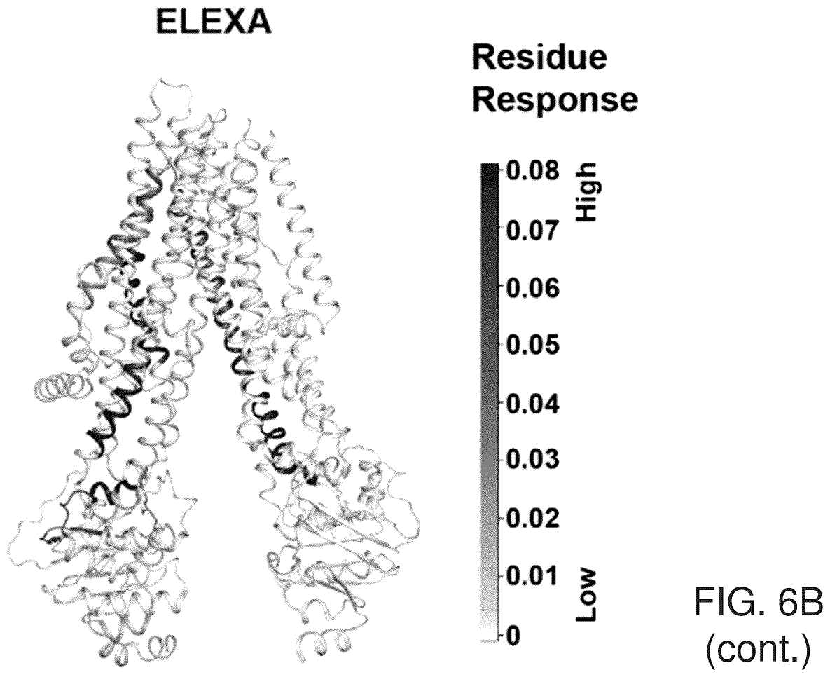

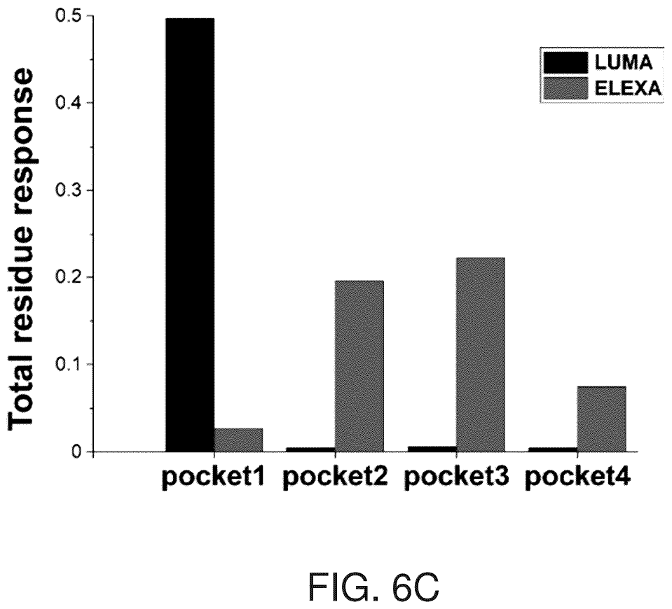

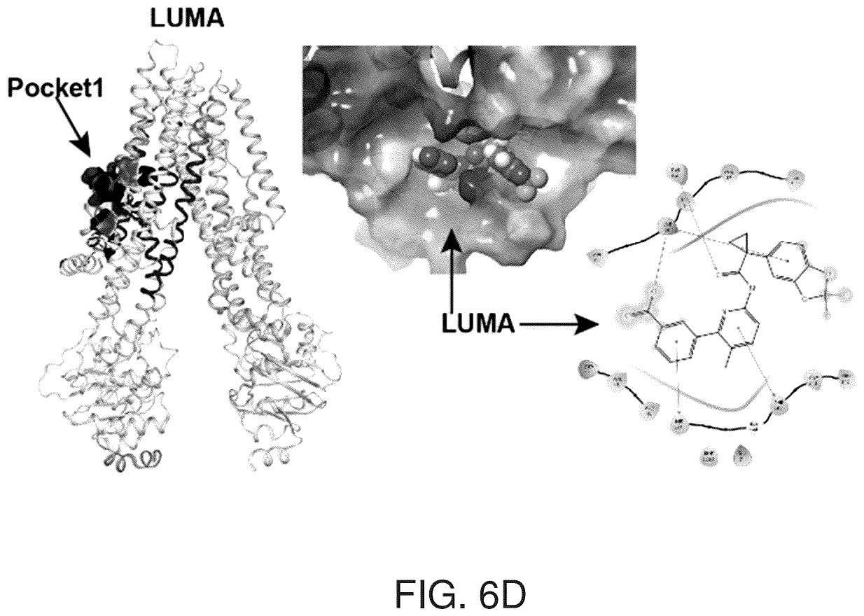

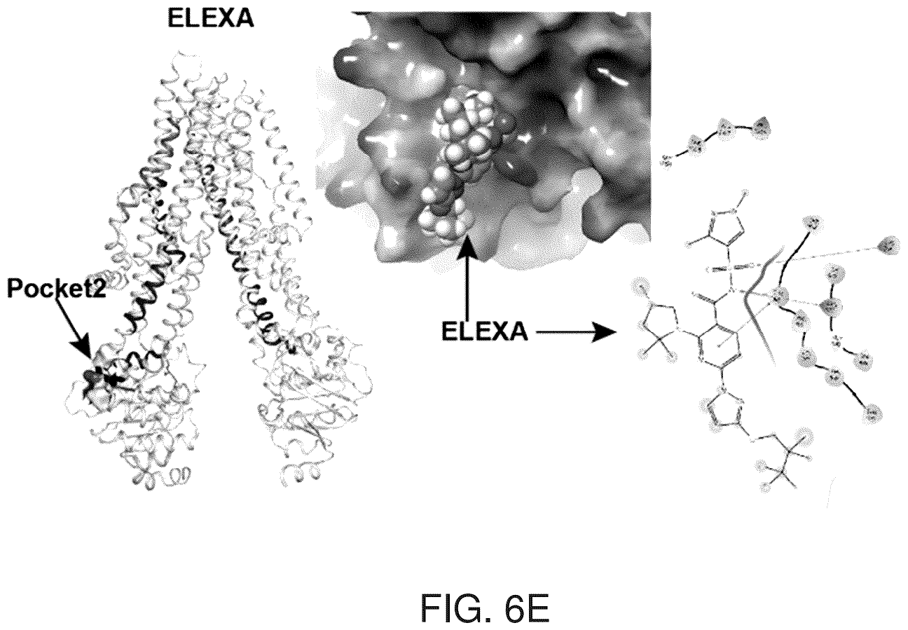

### Base Workflow Example 2—FIGS. 6A-6E

Referring now to FIGS. 6A through 6E, utility of SCVT tool/method for drug discovery through in-silico modeling is illustrated. In this example, sparse genetic variation in CFTR is used to predict the response of every residue in the entire CFTR polypeptide to two FDA approved drugs, Lumacaftor (LUMA) and Elexacaftor (ELEXA) from a sequence-based functional perspective, generating a first VarC map illustrated in FIG. 6A left panel with CFTR CICon response to LUMA as the z-axis, and second VarC map illustrated in FIG. 6B left panel with CFTR functional response to ELEXA as the z-axis (also represented by color). The right panel of FIG. 6A shows the on-diagonal covariance values from the VarC map on the left panel applied to the 3D CFTR model structure. Similarly, the right panel of FIG. 6B shows the on-diagonal covariance values from the VarC map on the left panel applied to the 3D CFTR model structure.

There are at least four binding pockets for LUMA and its analogue Tezacaftor that have been suggested by researchers through structural modeling studies. These suggested binding pockets are associated with particular residue clusters on the 3D structure of the CFTR protein. By associating the on-diagonal covariance values with the corresponding locations in the 3D structure model such as shown in the right panels of FIGS. 6A and 6B, residue clusters can be identified from the VarC map that show LUMA response and ELEXA response. This is illustrated in FIG. 6C. Overlap between residue clusters showing LUMA or ELEXA response and residues identified as possible binding pockets can determine the correct binding pocket associated with each therapeutic molecule. Based on the GRP-ML SCVT tool/method prediction, only the residues in pocket 1 respond to LUMA, demonstrating that it is the most likely binding pocket for LUMA. This result has recently been validated by the cryo-EM structure of the complex between LUMA and CFTR. Residues in pocket 2-4 do not respond to LUMA but show ELEXA responses, defined pocket for ELEXA binding. FIG. 6D illustrates pocket 1 containing a LUMA molecule, and FIG. 6E illustrates pocket 2 containing an ELEXA molecule. With the binding pocket(s) identified, drug discovery programs can be implemented targeting them with new small molecule therapeutics that may be more effective than existing therapeutics like LUMA and ELEXA in this example. These results validate that combining our SCVT tool/method linking sequence and function to structural modeling in-silico can identify drug binding pockets that provide an unprecedented SCVT based platform for therapeutic discovery.

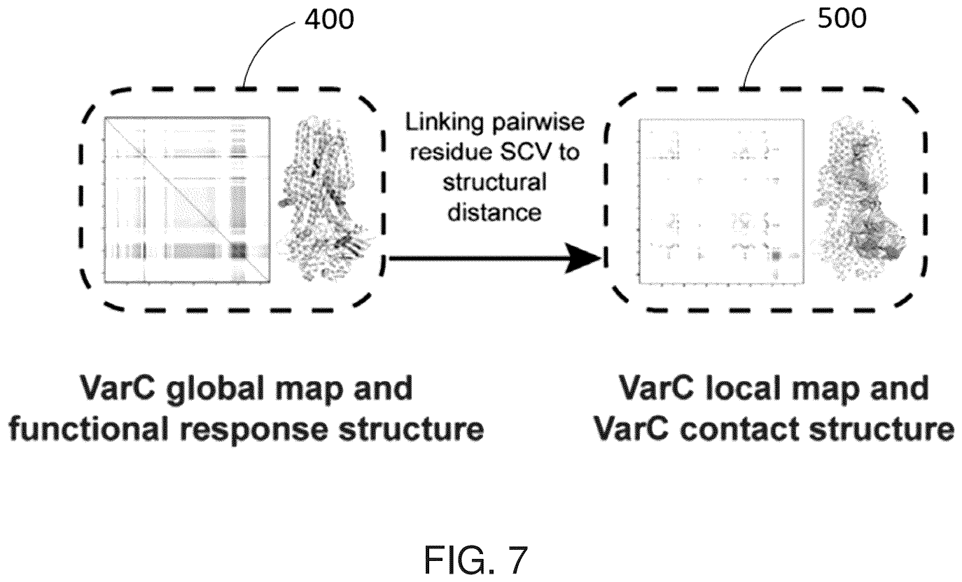

### First Extended Workflow Overview—FIG. 7

Referring now to FIG. 7, after a VarC map is created, pairwise-residue based atomic distance from an experimentally determined or computationally modeled 3D protein structure may be assigned to each pairwise-residue based functional SCV value on the off-diagonal of the VarC map to generate a molecular-distancing linked covariance (MDC) matrix to understand how the structural information is programmed to drive dynamic function. This is shown in block 500 of FIG. 7. Based on the MDC matrix bridging structural distance to functional covariance, the MDC map and the corresponding MDC structure can be built to illustrate how the residue-to-residue interactions connect local structural information to drive the short or long-range dynamic properties in the entire fold in response to physical and chemical perturbation. Although not illustrated in FIG. 7, the VarC map 400 may be generated via blocks 200 and 300 of FIG. 1 as discussed above.

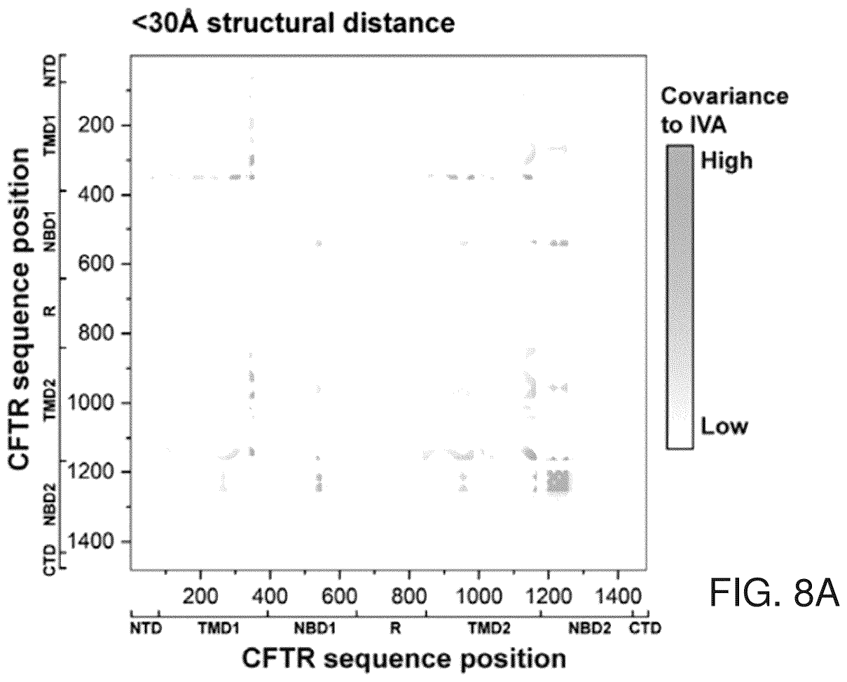

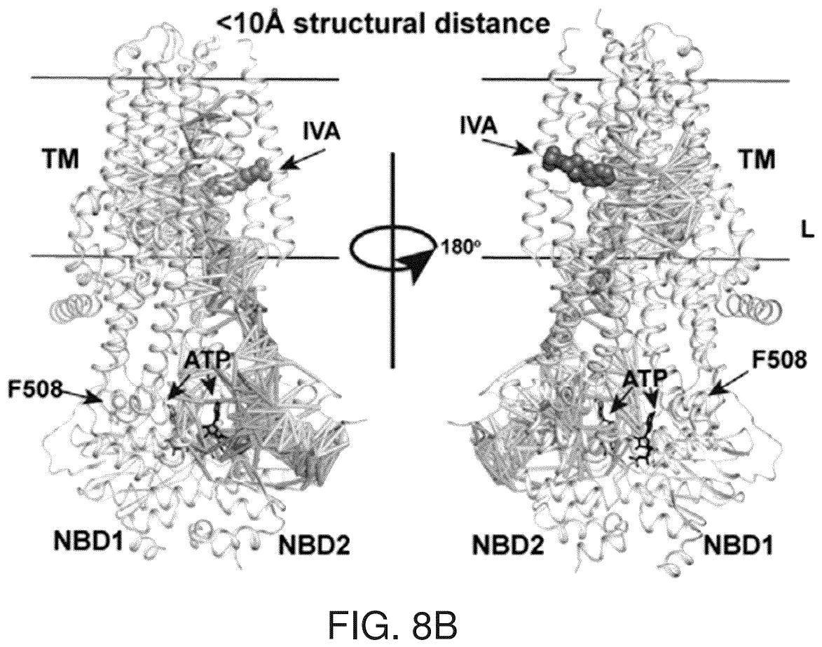

Turning now to FIGS. 8A and 8B, these Figures illustrate methods associated with block 500 of FIG. 7. The off-diagonal portions of the VarC map can be used to assess how the residue-to-residue (pairwise) covariance (i.e., off-diagonal signals) in the VarC global maps reflects 3D structure features in response to modulators, each pairwise-residue-based functional covariance value can be linked to the pairwise-residue-based atomic distance in the cryo-EM structure of the open conformation of CFTR. To focus on off-diagonal distal relationships contributing to the 3D fold, in some embodiments, residue pairs that are adjacent in sequence within five residues in linear sequence are excluded from the modified map, given the likelihood these residues are close in secondary structure. Since VarC does not use 3D structure as input to generate the functional covariance values, the correlation between the strong functional covariance relationships shown by VarC and their short 3D residue distances observed in structure demonstrates that linking linear sequence and phenotype information through VarC can capture otherwise unrecognized 3D structural features that contribute to protein function.

To generate the example MDC map of FIG. 8A, we compute the structure distance in the open conformation (PDB:6MSM) between the Ca atom for each residue pair. The reason why we choose the open conformation over the closed conformation in this example is the open conformation has more local structural contacts information than the closed conformation because NBD1 and NBD2 domains interact with each other in the open conformation but separate from each other in the closed conformation.

The covariance of residue pairs in VarC map that are within 30 Å structural distance are presented in the MDC map since most of the high response residue pairs have structural distance <30 Å. Furthermore, the long-range responsive residue pairs in 3D structure we observed for IVA treatment are also connected through the local residue pairs within 30 Å. Other proximity thresholds could be used, for example, any threshold from 10 to 50 Å.

To compare and visualize the different covariance matrices in response to different treatment, we first assign each of the covariance value in the 1480×1480 matrix a color code from white (RGB: 255, 255, 255) gradually to cyan (RGB: 0, 255, 255) or magenta (RGB: 255, 0, 255) or yellow (RGB: 255, 255, 0) (i.e., gradually decrease one color channel from 255 to 0) according to the covariance value in response to a treatment from low to high (scale from 0 to 0.04).

To generate the example MDC structure shown in FIG. 8B, residue pairs with Ca atom <10 Å were considered to be in contact since based on previous study (34) that a distance cutoff of 9 to 11 Å for Ca atom gives the optimal contact definition for reconstruction of contact maps to 3D structure. The color code of the corresponding covariance is assigned to the stick connecting these residue pairs within 10 Å distance in the structure using PyMol software. The sticks for the residue pairs with very weak or no responding covariance (covariance <0.005), i.e., whiter than color code (RGB: 223, 223, 223) are not shown.

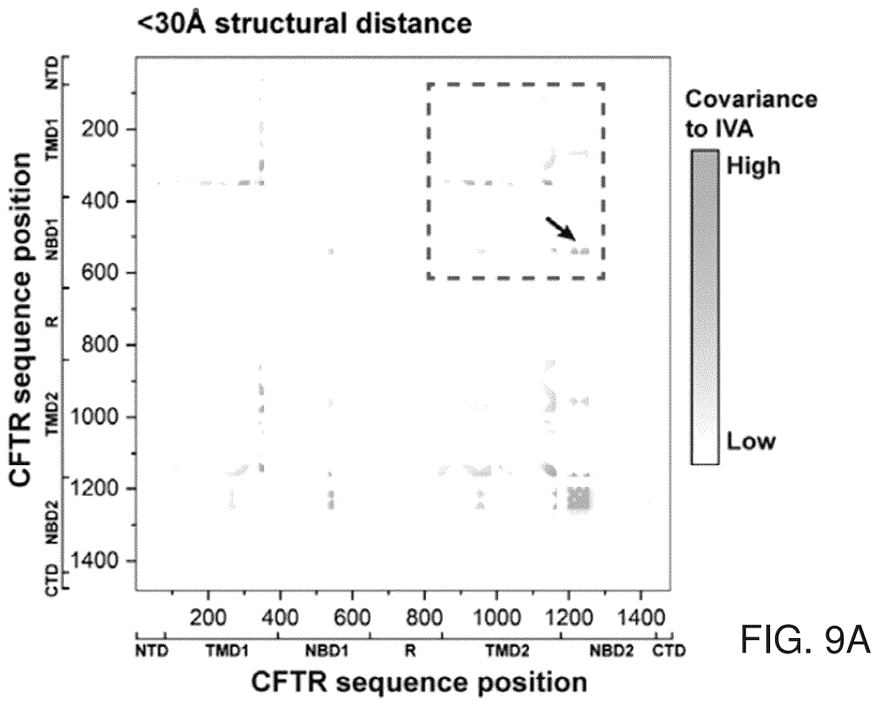

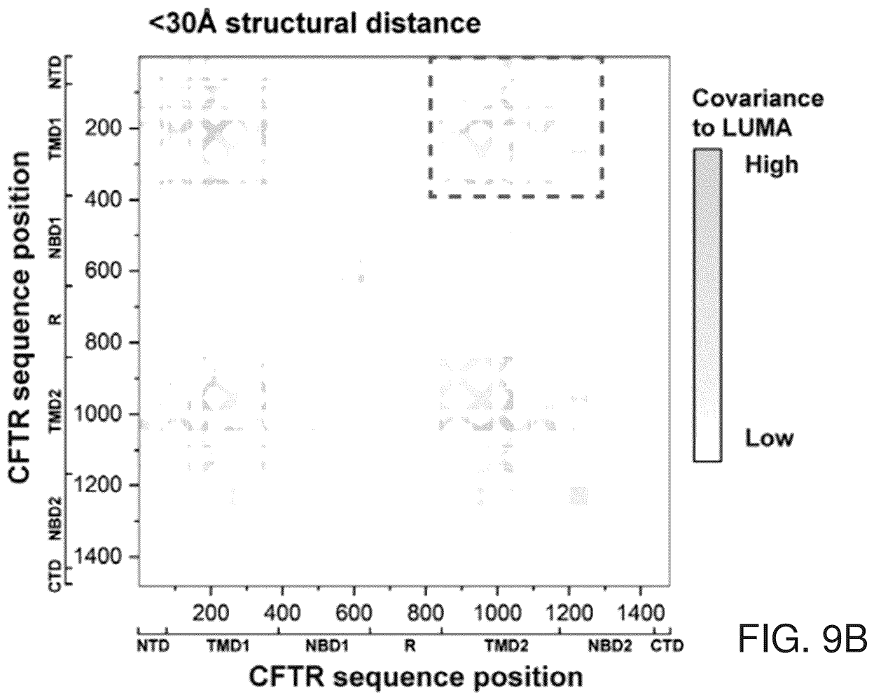

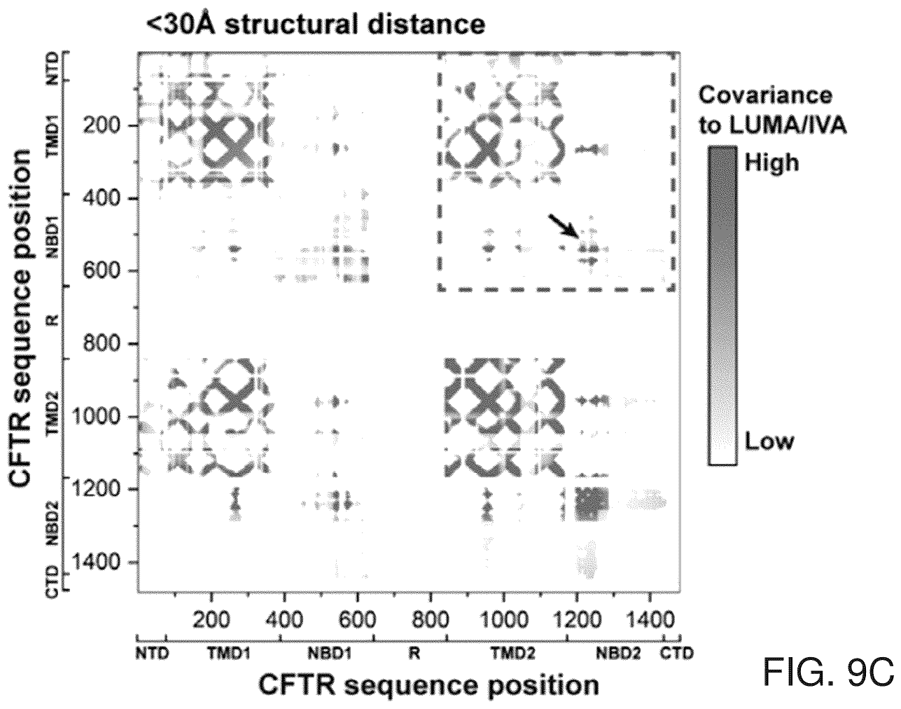

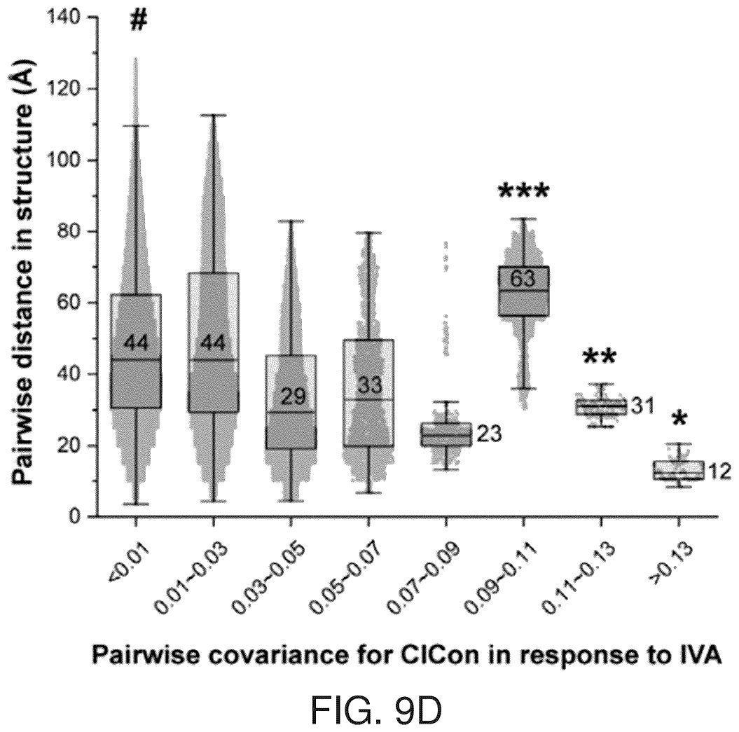

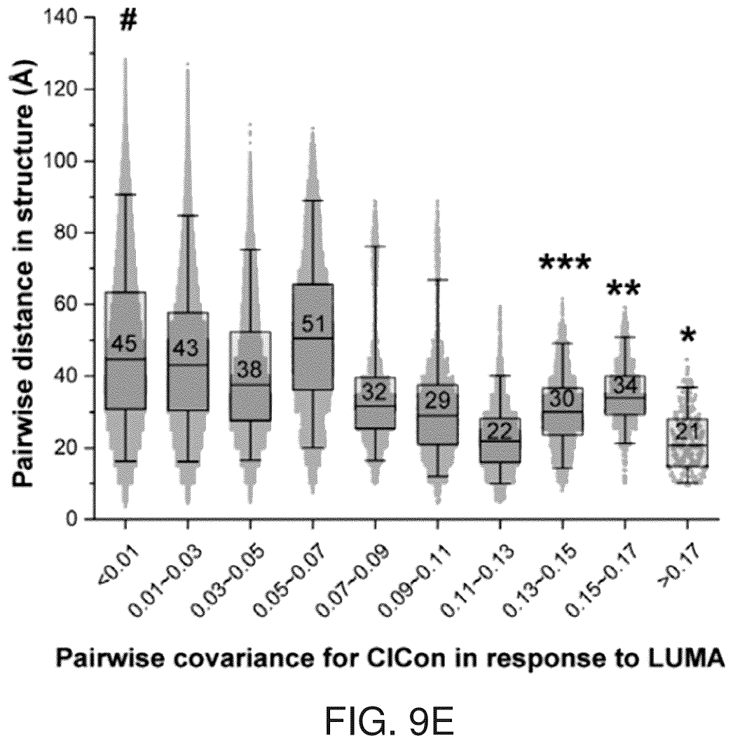

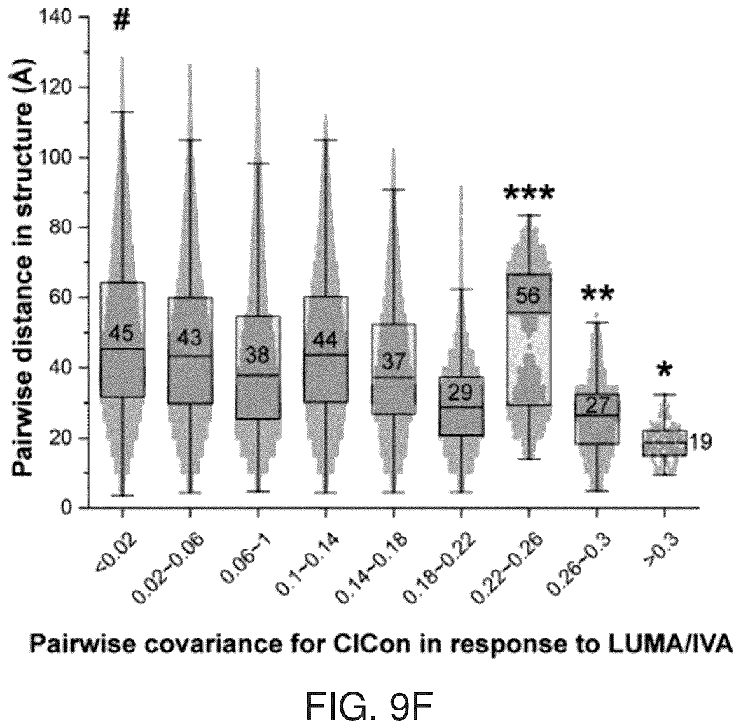

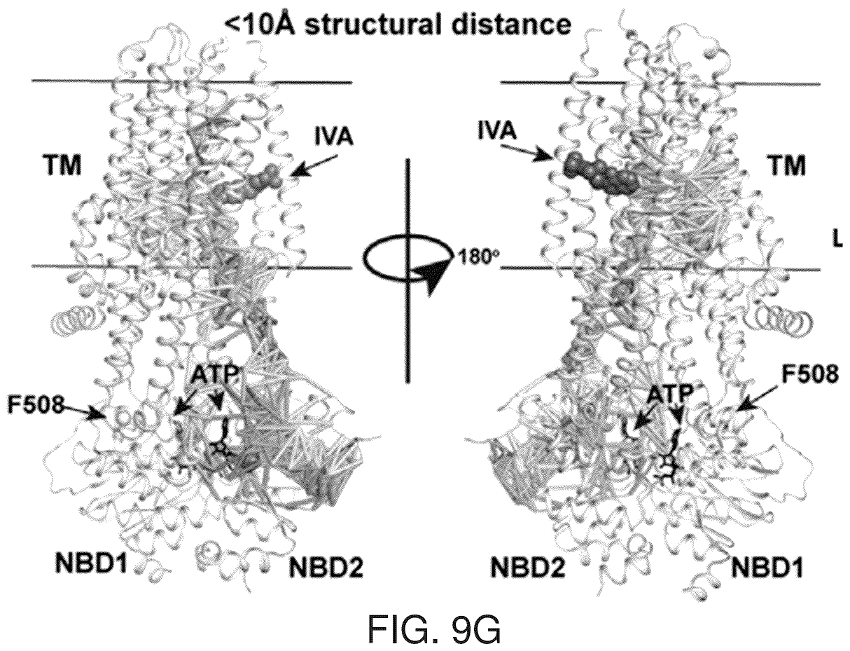

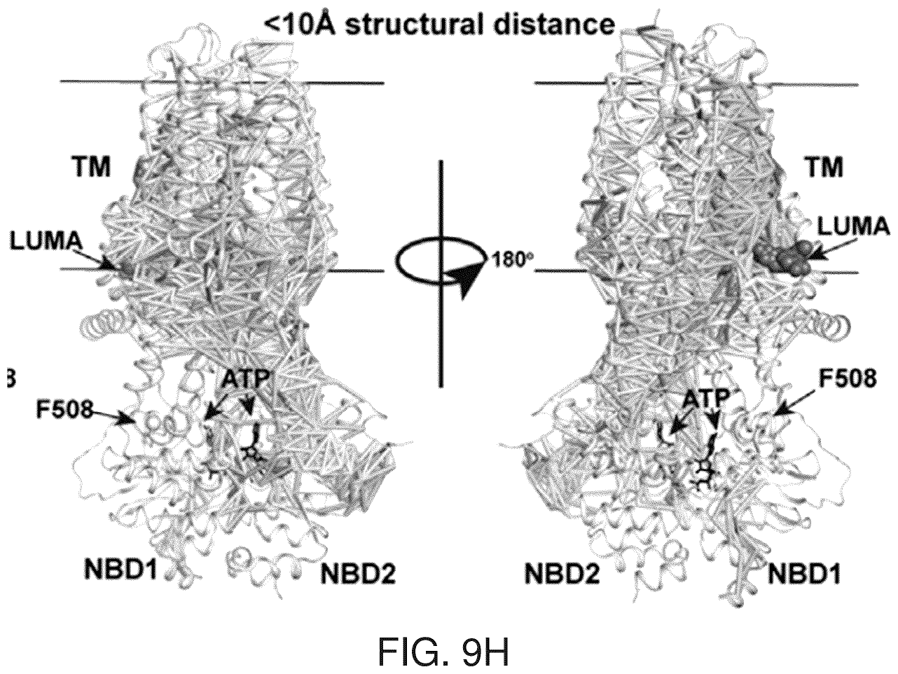

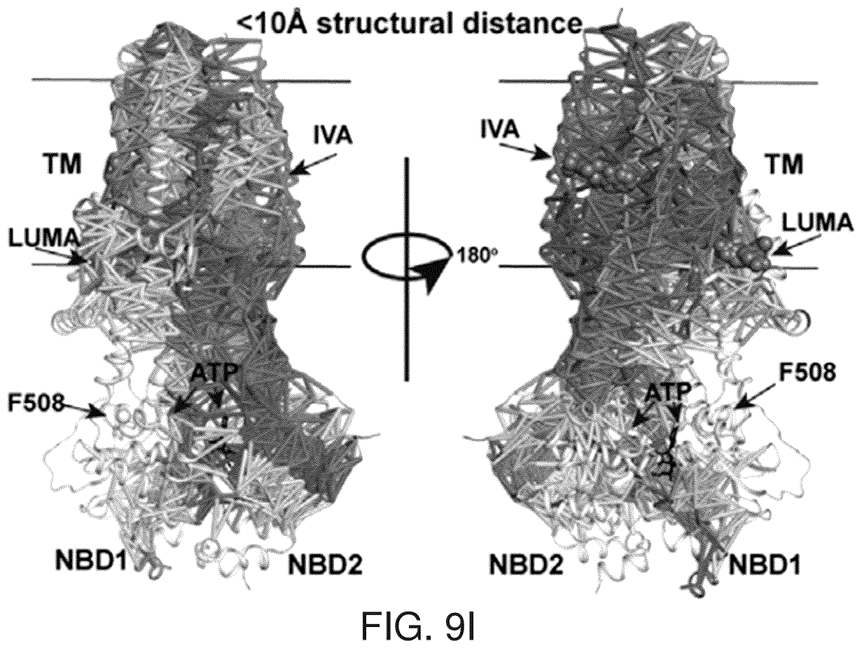

### First Extended Workflow Example 1—FIGS. 9A-9I

To assess how the residue-to-residue covariance (i.e., off-diagonal signals) in the VarC maps reflects 3D structure features in response to modulators, we generate an MDC matrix (FIG. 7, block 500) by linking each pairwise-residue based functional covariance value to the pairwise-based atomic distance in the cryo-EM structure of the open conformation of CFTR given it has contacts information between NBD1 and NBD2 domains. MDC maps for IVA, LUMA, and LUMA/IVA combo are shown in FIGS. 9A, 9B, and 90, respectively. We then binned the residue pairs in the MDC matrix according to their covariance response values (FIGS. 9D, 9E, 9F, x-axis) and plot the distribution of the pairwise-residue distance in 3D structure (FIGS. 9D, 9E, 9F, y-axis). To focus on off-diagonal distal relationships contributing to the 3D fold, we exclude any residue pairs that are adjacent in sequence within 5 residues in linear sequence given the likelihood these residues are close in secondary and 3D structures. While the overall median distance for non-responding covariance residue pairs to modulators is 44˜45 Å in the CFTR structure (FIGS. 9A, 9B, 9C), the highest and the second highest groups of responding residue covariance pairs are in spatial proximity with median distance ˜12-21 Å and ˜27-34 Å respectively for different modulator treatments, illustrating the local structural relationships used to form the high functional covariance response. Interestingly, LUMA and IVA trigger different off-diagonal structural patterns to drive CFTR CICon function (FIG. 9A vs. 9B). For example, the bin with the third highest covariance in response to IVA (FIG. 9D, x=0.09˜0.11) has a median distance value at 63 Å in 3D structure, revealing that long range structural dynamics are involved in the IVA response (FIG. 9A). In contrast, the major responding residue pairs to LUMA are more clustered with a median structural distance from 20 Å to 30 Å (FIG. 9B). In LUMA/IVA mix we see a combined pattern of LUMA and IVA (FIG. 9C). These results suggest that the functional SCV information encoded by the genome can flexibly reprogram many local structural information relationships to achieve the short or long-range structural dynamics that drive CFTR CICon function in response to different chemical environments.

To understand how the direct physical residue-residue interactions in 3D structure supports the functional SCV relationships, we generated MDC structures based on the MDC map by projecting the functional covariance value of residue pairs that are in physical contact using 10 Å distance of Cα atom on CFTR structure as a cutoff. These are shown in FIGS. 9G, 9H, and 9I. The MDC structures reveal pairwise residue-based SCV relationships for CICon response to IVA (FIG. 9G, cyan color), LUMA (FIG. 9H, yellow color) and LUMA/IVA (FIG. 9I, magenta color) that show the plasticity involved in therapeutic management of the functional fold in the cell.

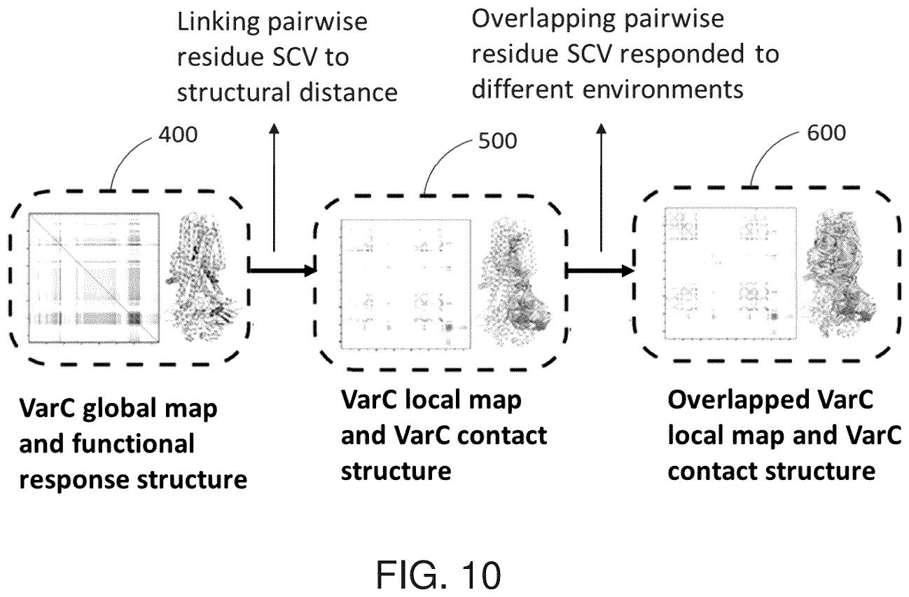

### Second Extended Workflow Overview—FIG. 10

A second extended workflow is illustrated in FIG. 10. In this Figure at block 600, different SCV based MDC maps and/or structures may be overlapped to quantitatively characterize the molecular mechanisms triggered by differential modulation of physical and chemical environments across the entire protein to shape function in human health and disease. When different color MDC matrices (or structures) are overlapped, the color channels described above from the different VarC maps will be combined. For example, if the same residue pair is highly responding (e.g., covariance >0.04) in both yellow (RGB: 255, 255, 0) and magenta (RGB: 255, 0, 255), the combined color will be red (RGB: 255, 0, 0). Although not illustrated in FIG. 10, the VarC map 400 may be generated via blocks 200 and 300 of FIG. 1 as discussed above.

### Second Extended Workflow Example 1—FIGS. 11A-11B

This Example refers back to the MDC matrices of FIGS. 9A, 9B, and 9C and MDC structures of FIGS. 9G, 9H, and 9I. To directly compare these different structural covariance matrices in response to modulator treatment, overlapped MDC maps and corresponding MDC structures were generated for IVA and LUMA (FIG. 11A). The direct comparison clearly reveals unanticipated residues in the TM region that only respond to LUMA to facilitate the improvement of CFTR function (FIG. 11A, MDC matrix, yellow). Furthermore, the structural SCV relationships through the channel region linking NBD1 and NBD2 are mainly targeted by IVA (FIG. 11A, MDC matrix, cyan), though some are also partially impacted by LUMA (FIG. 11A, MDC matrix, green).

To define the residues impacted by the LUMA/IVA mix over those impacted separately by LUMA or IVA alone, we overlapped the three MDC maps and MDC structures for IVA, LUMA and LUMA/IVA (FIG. 11B). The overlap shows that the LUMA/IVA mix retains all the responses for IVA and LUMA, but also reveals enhanced response for many residues affected by IVA and LUMA combination. These enhanced activities include cell surface localized variants and the region around IVA binding site. In striking contrast, the most common CF-causing mutation F508del (found in ˜90% of patients) and the interacting residues with F508 have almost no or little response to IVA, LUMA or LUMA/IVA treatment (FIGS. 11A and 11B, arrows labeled F508). Surprisingly, variant based SCV relationships, quantitatively defined by VarC mapping across the entire polypeptide sequence, reveals that neither LUMA and IVA alone, nor the LUMA/IVA mix, mechanistically directly target the residue-to-residue SCV relationships connected to F508del. VarC analysis challenges the concept that LUMA is a ‘corrector’ for F508del, having almost no or little impact on the residue-to-residue interactions required to rescue the F508del fold for export from the ER, explaining the poor response of patients in the clinic.

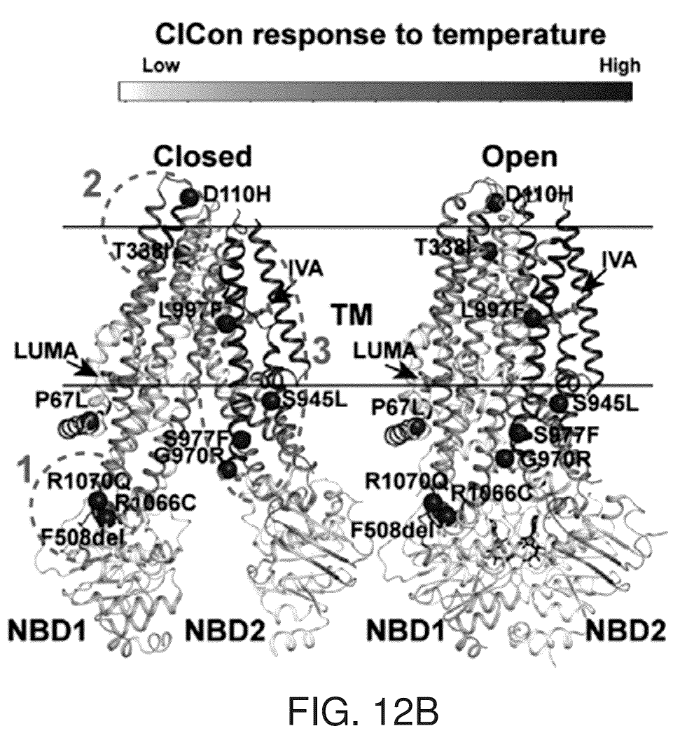

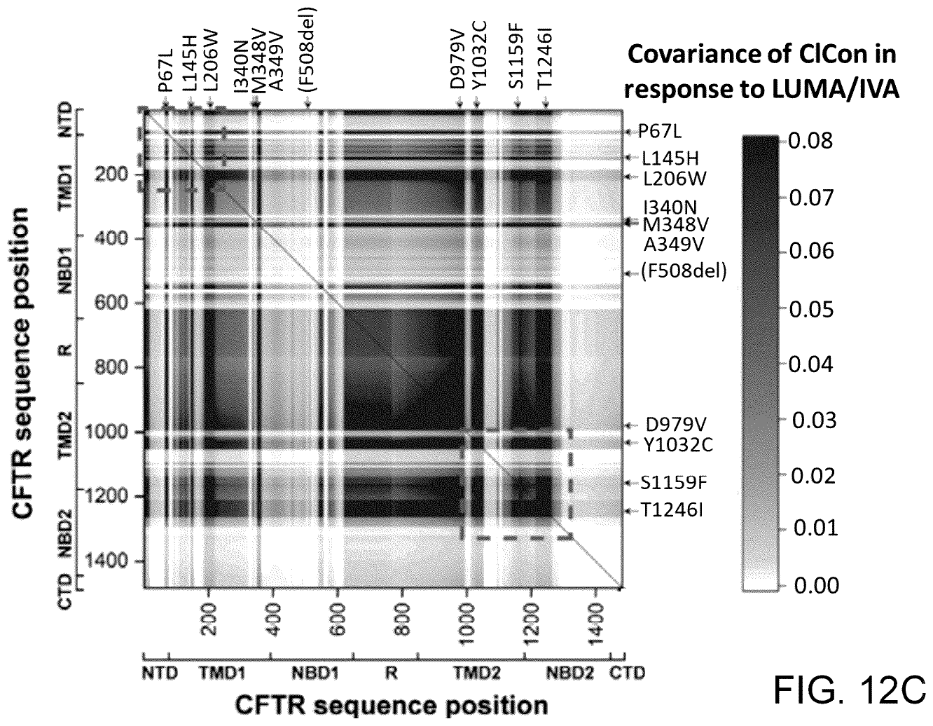

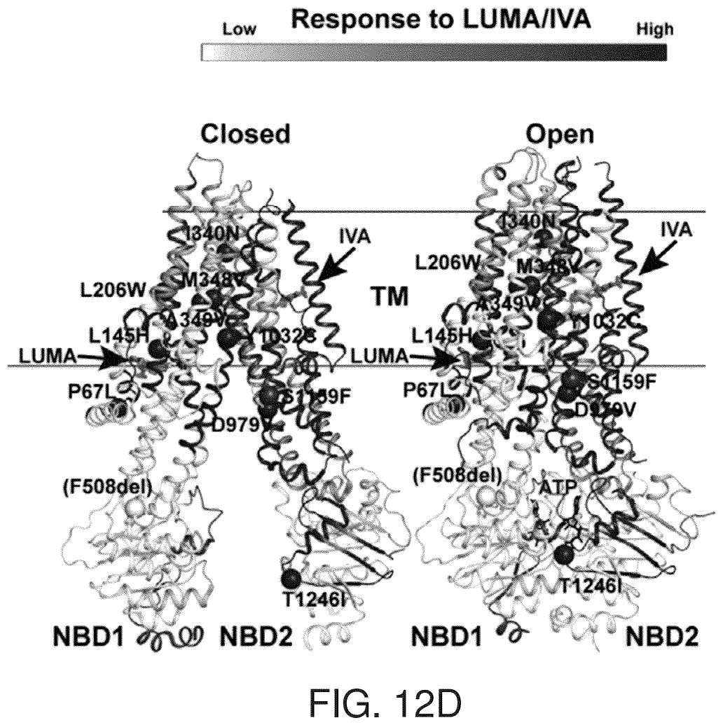

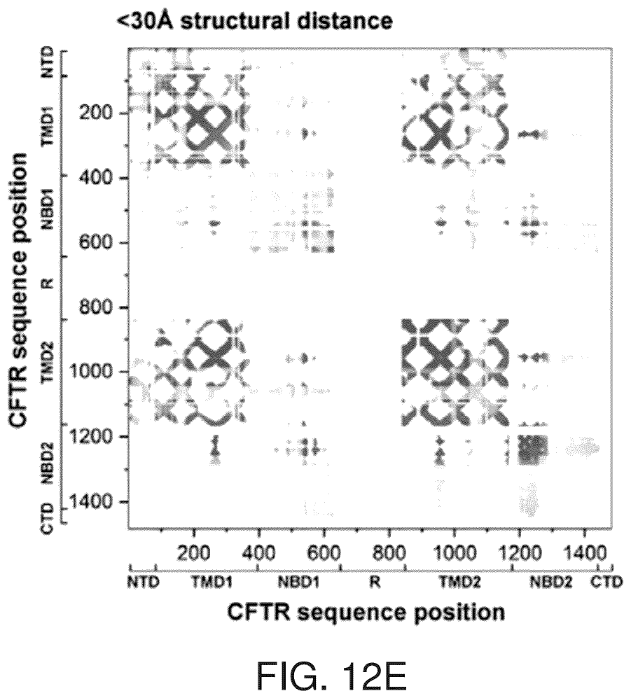

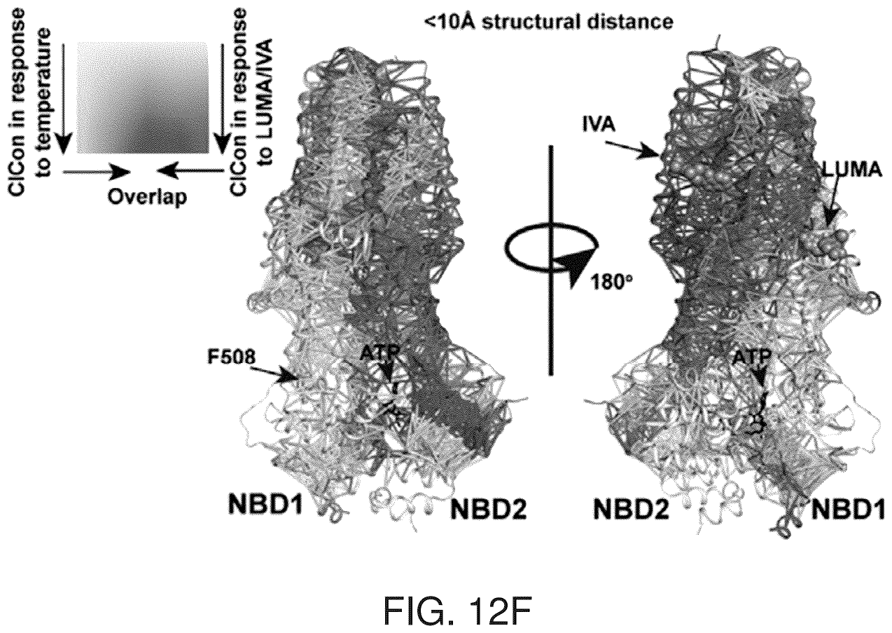

### Second Extended Workflow Example 2—FIGS. 12A-12F

Given that transformation from the genotype to the phenotype is intimately linked to the energetics in biology, what are the fundamental SCV based fold energetics that are completely missed by LUMA/IVA mix but could contribute to improved management of CF, or any disease using our VarC approach? To address this fundamental concern, we extended the well-established observation that temperature shift from 37° C. to 27° restores the trafficking and CICon function of F508del to our collection of variants at 27° C. Here, cells were incubated for 24 h at 27° C. and specific activity of CICon measured. A temperature shift (TS) CICon-phenotype landscape predicting all possible SCV relationships in the polypeptide sequence was used to build the corresponding pairwise-residue based TS/CICon-VarC map (FIG. 12A). We then mapped the known and predicted on-diagonal values of the TS/CICon-VarC map to the closed and open conformations of CFTR to generate a TS/CICon-functional-response-structure (FIG. 12B). The TS/CICon-functional-response-structure reveals 3 major structural regions where the CICon function of variants are highly corrected by low temperature at 27° C., including: 1) F508del in NBD1 and its interaction with intracellular loop 4 (ICL4), 2) extracellular loop regions, and 3) regions around IVA binding sites (FIG. 12B, red dash circles). These results reveal critical energetically sensitive properties of the fold promoting the plasticity responsible for CICon.

The overlapped MDC map and structure (FIGS. 12E and 12F) between TS and LUMA/IVA treatment (FIG. 12C) show that they have both shared (FIG. 12E, red) and unique (FIG. 12E, yellow or magenta) pairwise-residue based covariant responses. For example, the residue interactions surrounding IVA binding site can be functionally resolved by both temperature and LUMA/IVA treatment (FIG. 12F, red), consistent with the role of IVA as potentiator favorably altering the folding energetics to afford CICon through the channel. On the other hand, F508 and its pairwise-residue interactions, that can be corrected by temperature shift, are negligibly corrected by the LUMA/IVA mix (FIG. 12F, yellow) suggesting that the SCV relationships impacting the energetic features in this region that are critical for export and trafficking to the cell surface are not impacted by the LUMA/IVA mix.

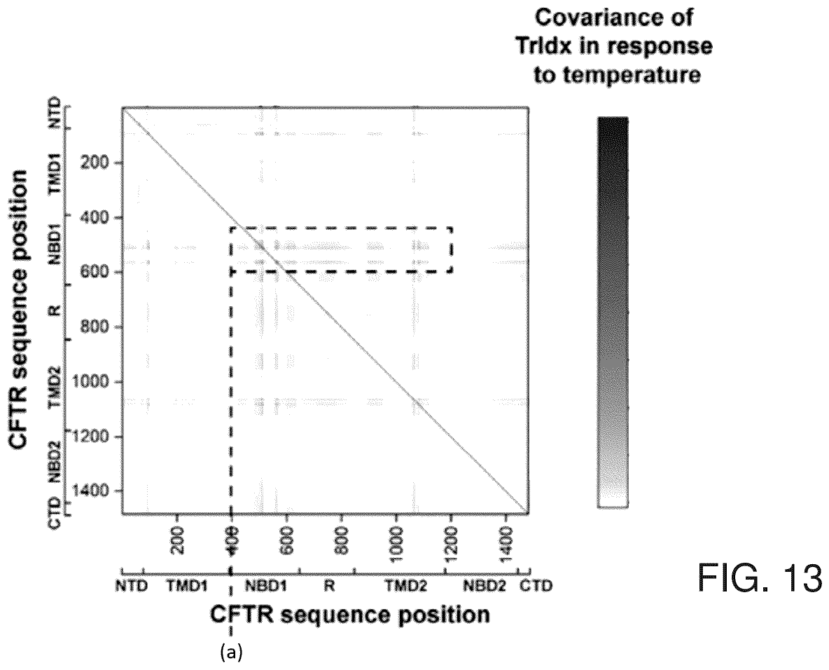

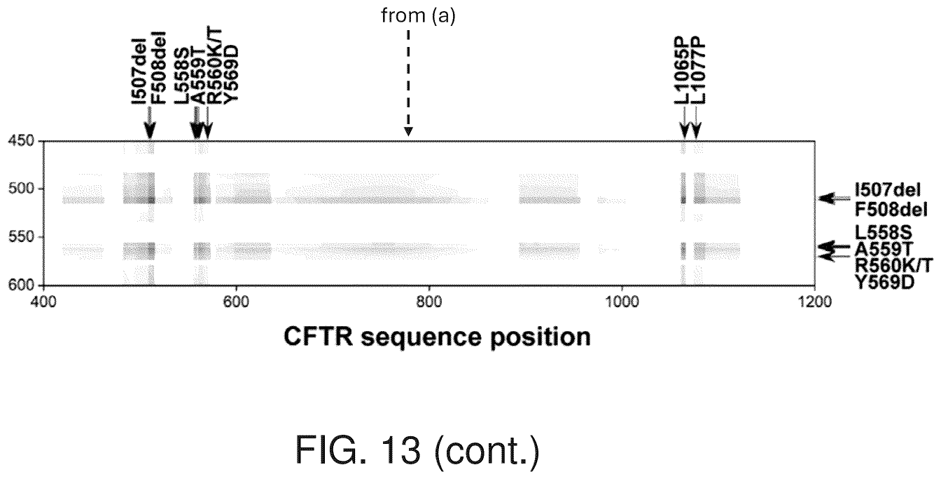

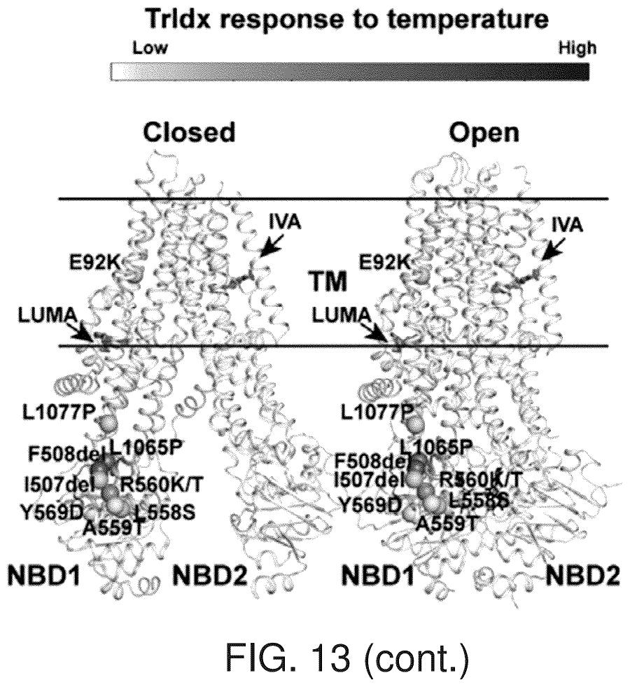

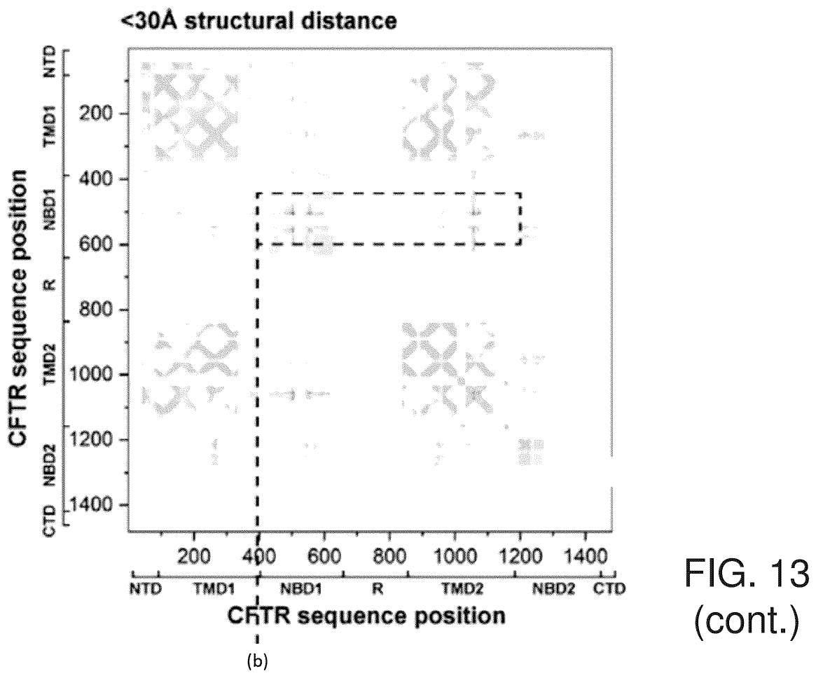

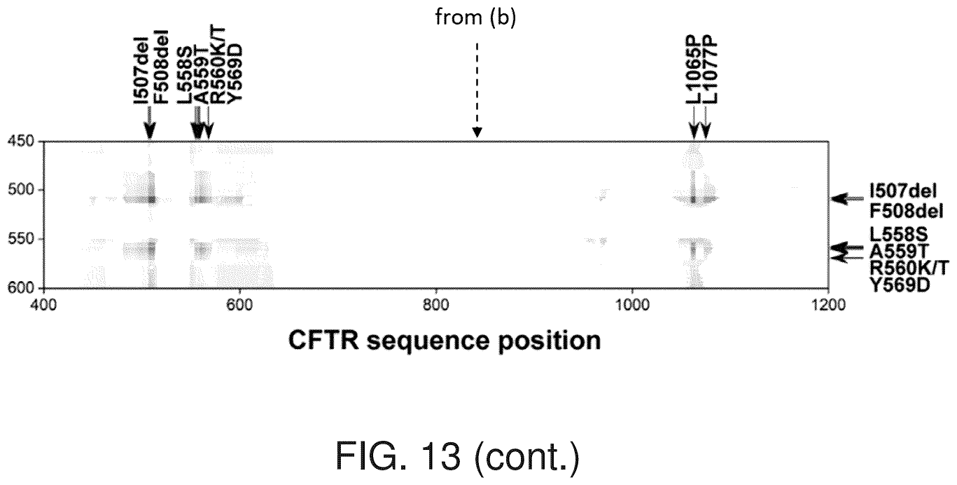

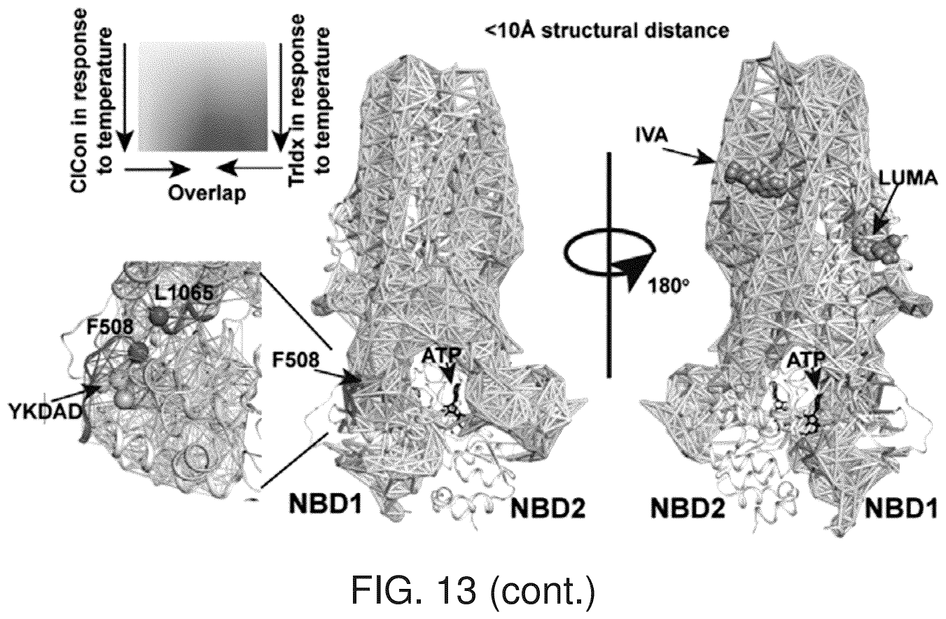

### Second Extended Workflow Example 3—FIG. 13

Referring now to FIG. 13, to address whether the correction of CICon in response to temperature is due to energetic features associated with global SCV relationships responsible for membrane trafficking of CFTR from the ER, we built the VarC map for the CFTR trafficking index (TrIdx) of variants following shift from 37° C. to 27° C. TrIdx quantifies the amount of CFTR in post-ER compartments relative to total CFTR in the cell as a measure of efficiency of ER export. The pairwise-residue based TS/TrIdx-VarC map (FIG. 13, upper left panel) and its corresponding TS/TrIdx-functional-response-structure (FIG. 13, upper second from left panel) reveal that only a limited subset of sequence residues contributes to TrIdx correction upon temperature shift. These include: 1) a sequence region containing F508 (FIG. 13, upper left panel, inset zoom); 2) a region in NBD1 domain including variants L558S, A559T, R560K/T and Y569D that are surrounding the CFTR di-acidic code (YKDAD (residues 563-567)) required for COPII dependent ER export (FIG. 13 upper left panel, inset zoom); 3) a sequence region in ICL4 that is reported by CF variants L1065P and L1077P (13) (FIG. 13, upper left panel, inset zoom). Even though separated in primary sequence, these responding regions cluster together in structure (FIG. 13, upper second from left panel). Overlap of MDC maps and corresponding MDC structures between TS/CICon and TS/TrIdx identifies a common energetic sensitive core (FIG. 13, right side, red) responding to temperature shift for both TrIdx and CICon function. This SCV based energetic sensitive core contains the F508-L1065 covariant link that bridges NBD1 (F508) and ICL4 (L1065) (FIG. 13, right panel, inset zoom, red), mechanistically explaining the previous observation that the NBD1-ICL4 interaction is crippled by F508del. While TrIdx for the YKDAD region is significantly improved in response to temperature shift (FIG. 13, right panel, inset zoom, magenta), CICon is not, reflecting distinct quality systems between the ER and cell surface. The remarkably compact design of the temperature sensitive core responsible for delivery of CFTR for function at the cell surface suggests the existence of an evolutionarily fine-tuned energetic cluster specifically managing information flow from the genome sequence to polypeptide fold design.

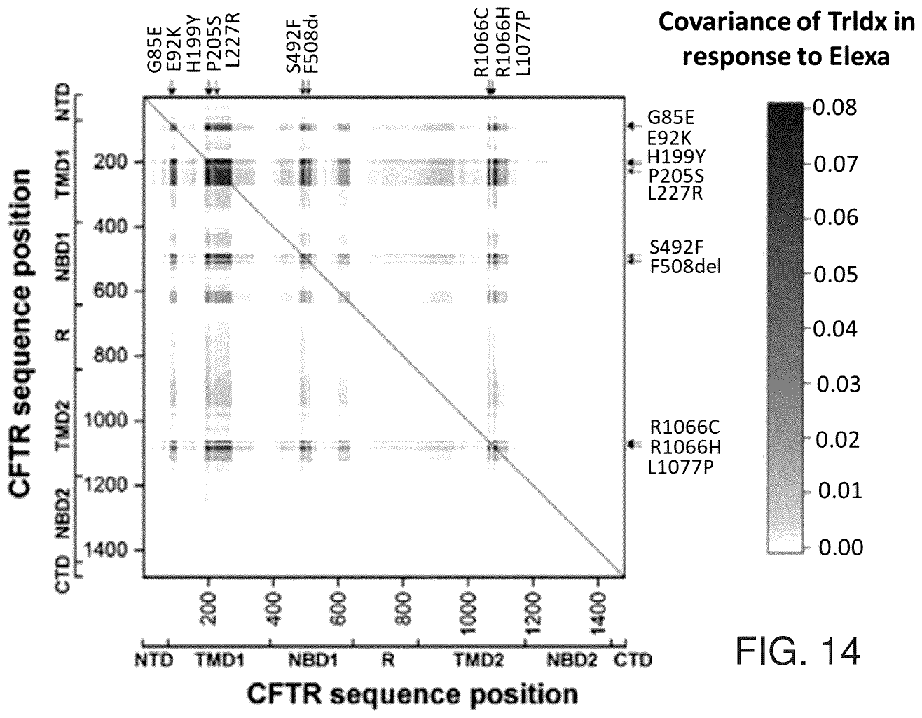

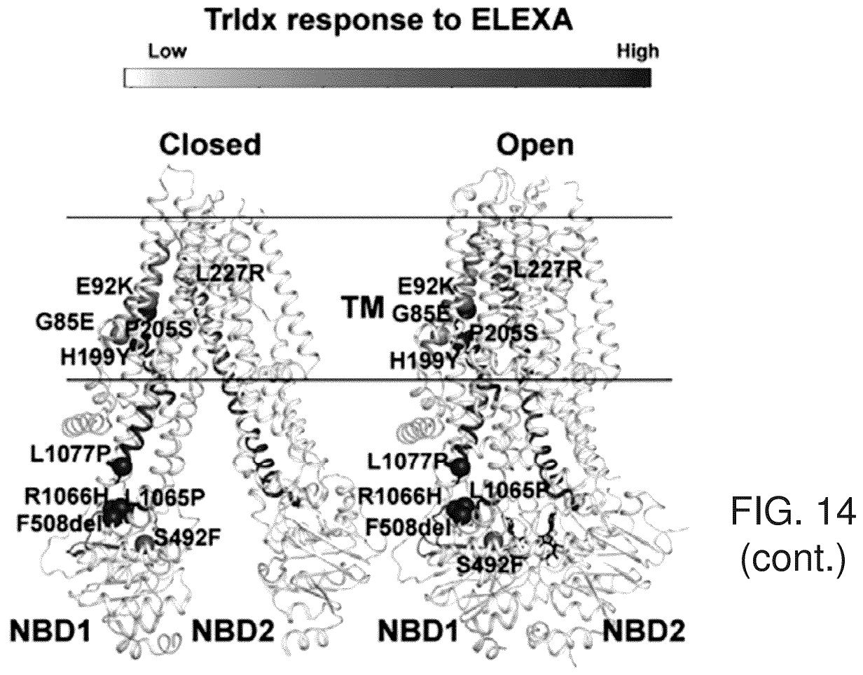

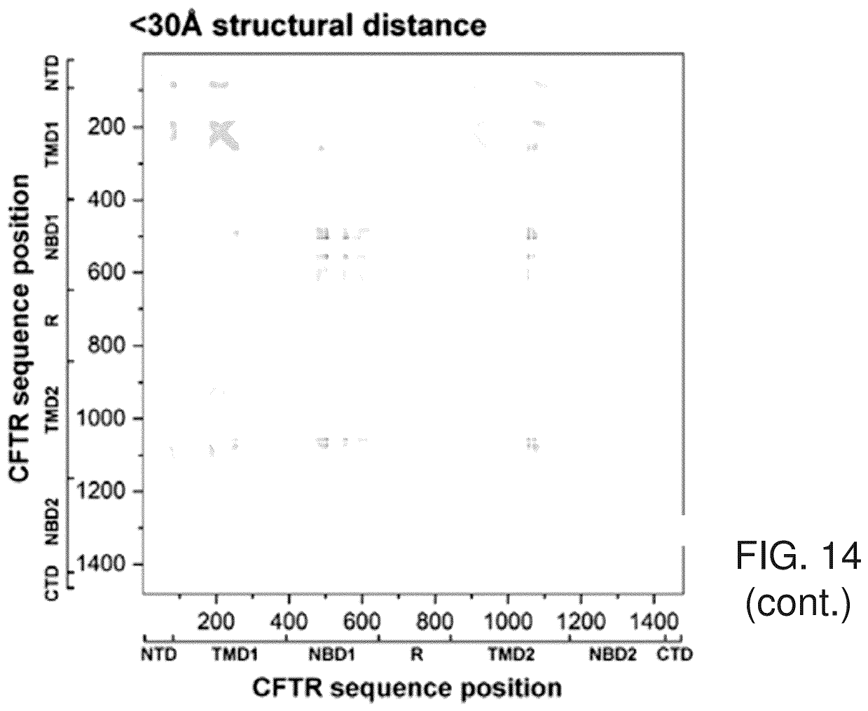

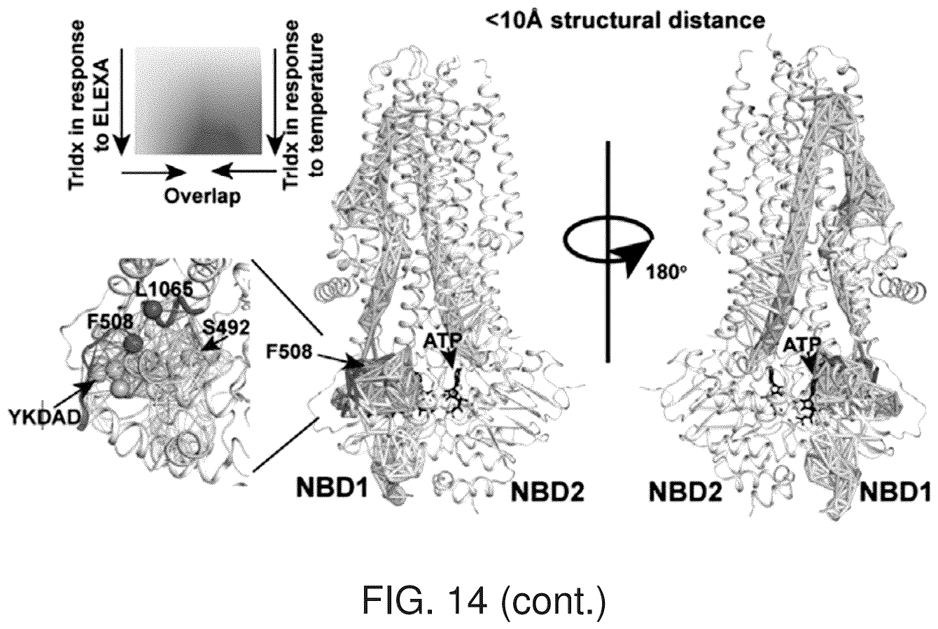

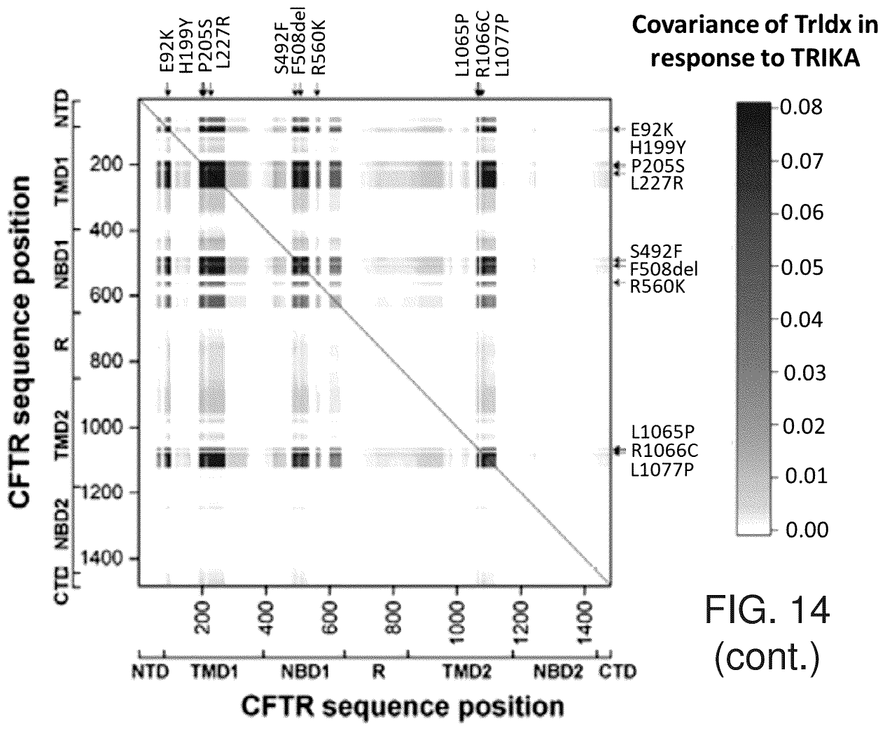

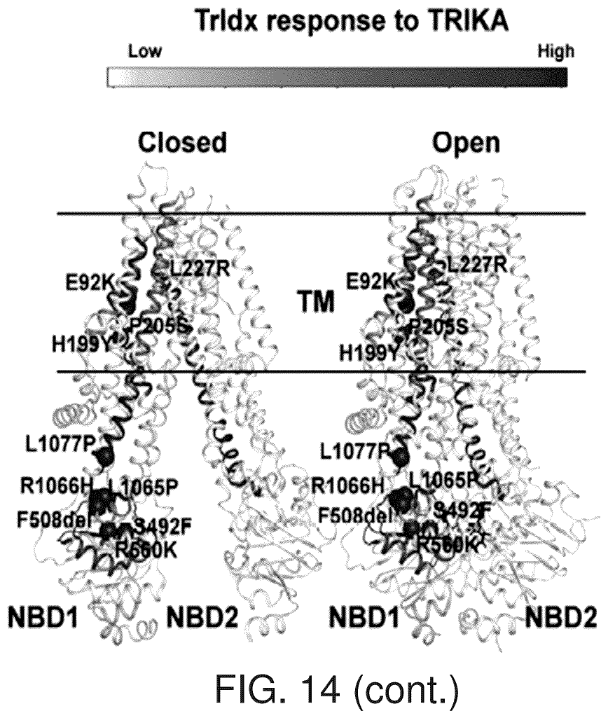

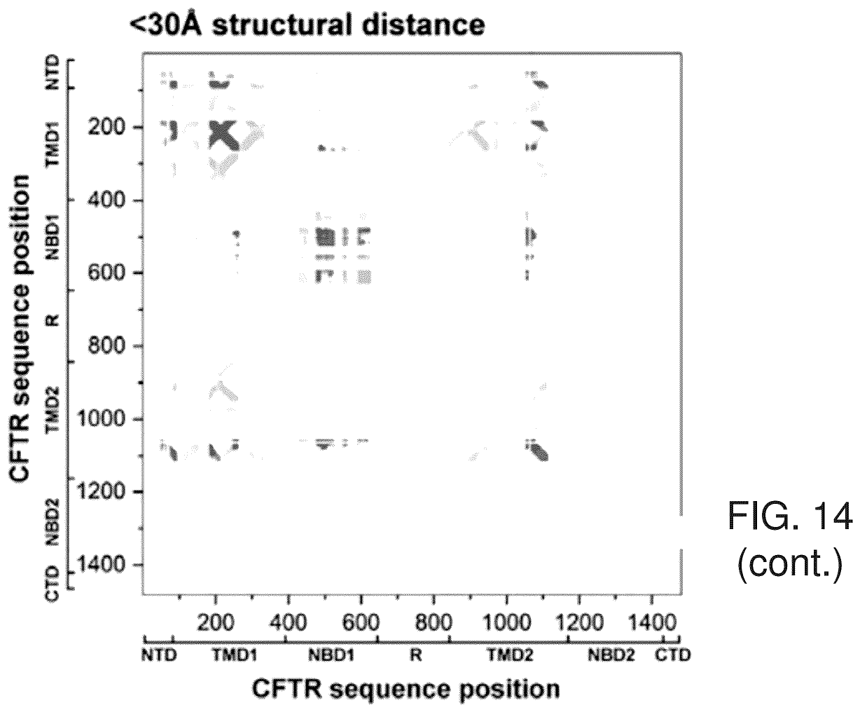

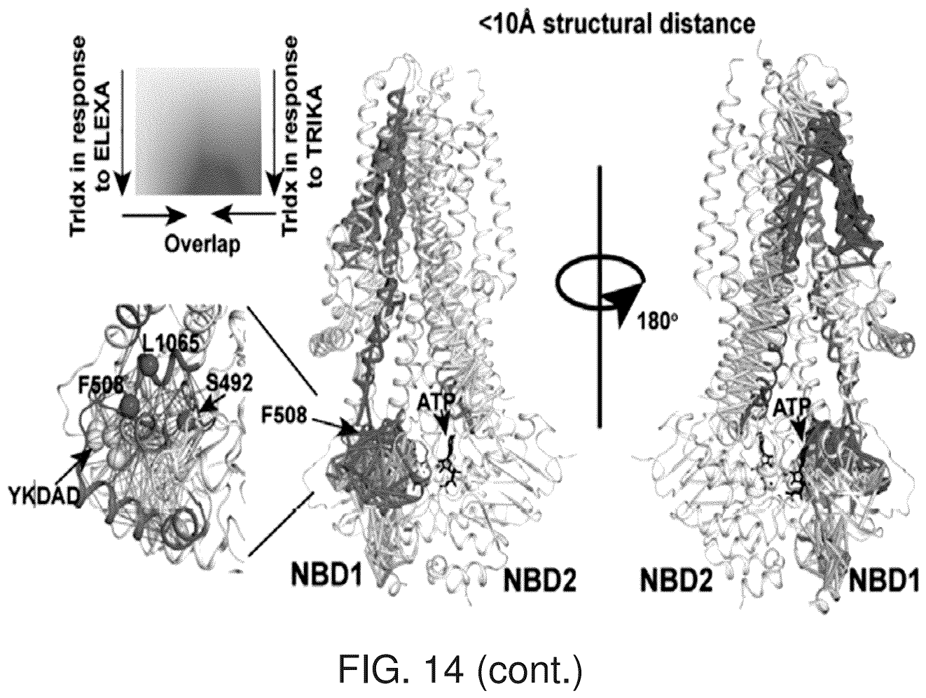

### Second Extended Workflow Example 4—FIGS. 14 Through 16

The limited correction of the energetic core by LUMA/IVA for the vast majority of CF patients harboring F508del prompted us to investigate the impact of ELEXA and its triple combination with TEZA and IVA (TRIKA) that has demonstrated considerably improved efficacy in the clinic relative to the LUMA/IVA mix. Consistent with previous results, using F508del-CFBE41o− cells, a bronchial epithelial cell line stably expressing the F508Del CFTR transgene, we found ELEXA alone can increase both the total level and the trafficking efficacy of F508del. Combination of ELEXA and TEZA led to synergistic improvement of both the total level and trafficking of F508del when compared with the impact of ELEXA alone. A detailed kinetic study of ELEXA and TEZA shows that the impact of ELEXA/TEZA combination on trafficking and total F508del level is dominated by ELEXA that works as a pharmaceutical chaperone. Adding IVA to ELEXA has no additional impact on TrIdx. IVA in combination with ELEXA/TEZA (TRIKA) slightly decreases both the total level and TrIdx of F508del when compared with that of ELEXA/TEZA, possibly due to a small antagonistic effect of IVA and LUMA on protein processing.

To address how ELEXA and TRIKA manage SCV relationships for the entire CFTR polypeptide for ER export, we measured the TrIdx for 64 CFTR variants in response to ELEXA and TRIKA treatment, which show diverse responses for different variants. We next explored the underlying SCV based rules that describe the global impact of ELEXA and TRIKA on the fold as we did for LUMA and IVA. We first build a TrIdx-VarC map in response to ELEXA (FIG. 14, upper left panel) using the variant-based TrIdx-phenotype landscapes SCV predictions for ELEXA. The ELEXA/TrIdx-VarC map reveals highly responsive sequence regions in NTD, TMD1, NBD1 and TMD2. Visualization of the response on the corresponding ELEXA/TrIdx-functional-response-structure now shows a responding 1D sequence region encoded by the genome in the NTD that interacts with a sequence region in TMD1 that in-turn allosterically links to F508del in NBD1 through the responding residues in TMD2 in the 3D fold, suggesting that ELEXA improves cooperative folding events between the transmembrane region and NBD1.

To compare the pairwise-residue covariance differences in the TrIdx response to either ELEXA or temperature shift, we overlapped their respective MDC maps and MDC structures. These results reveal for the first time that the critical bridge (F508-L1065) linking NBD1 with ICL4 (unlike that observed for the LUMA/IVA mix) can be corrected by both ELEXA and temperature shift (FIG. 14, upper right, inset zoom, red), although the highly responsive TMD1 region targeted by ELEXA is not influenced by low temperature (FIG. 14, upper right, yellow). Critically, the essential di-acidic ER export code (YKDAD-563-567) region in NBD1 corrected by reduced temperature is not corrected by ELEXA (FIG. 14, upper right, magenta), indicating mechanistic differences in the response of CFTR variants to ELEXA and energetic rules dictating the fundamentals of information flow in CFTR genome to proteome design.

To understand how TRIKA drives correction of F508del from a global SCV perspective, we generated TRIKA/TrIdx VarC map and the TRIKA-TrIdx response structure (FIG. 14, lower left), which show a stronger and broader impact when compared with the ELEXA response alone. A direct comparison of the MDC maps and MDC structures between ELEXA and TRIKA clearly indicates there are many pairwise-residue interactions in the NBD1 domain that are only improved by TRIKA. Again, the di-acidic ER export code (YKDAD) has very limit response to TRIKA. These results demonstrate that TRIKA does not fully correct the SCV based pairwise-residue cluster that is critical for the stability of the fold required for ER export and function at the cell surface.

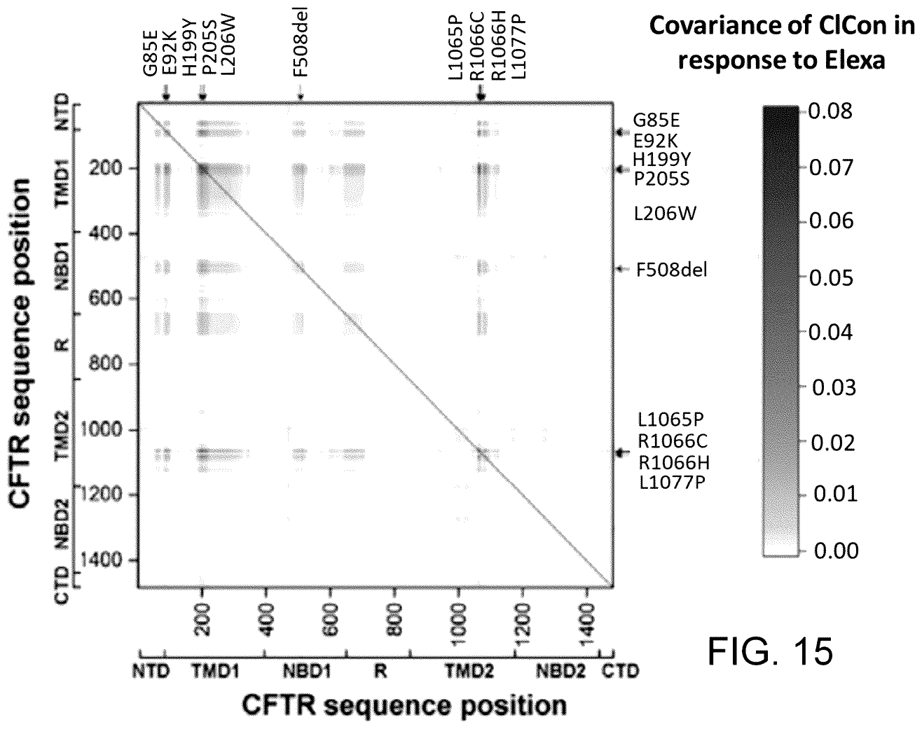

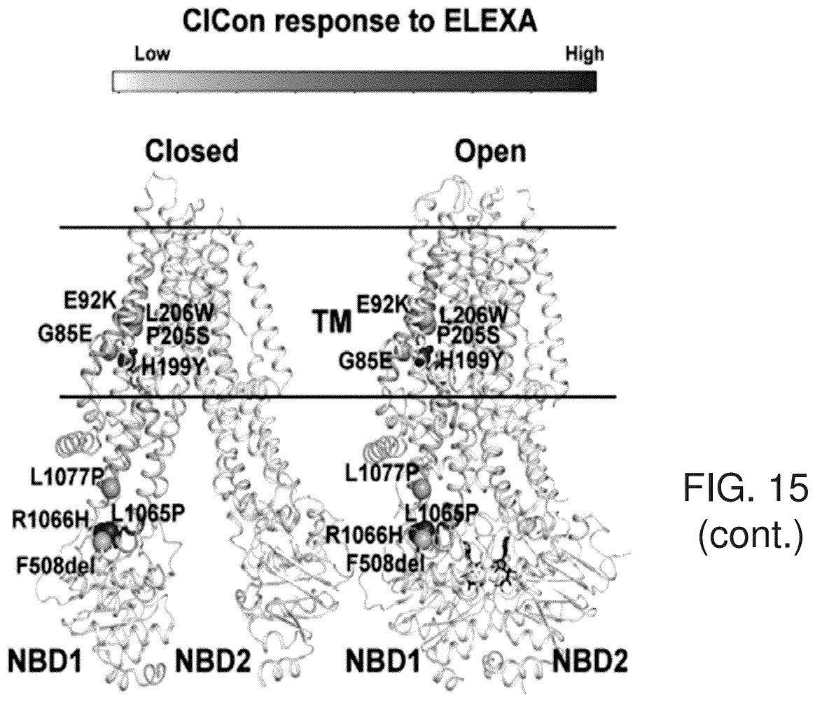

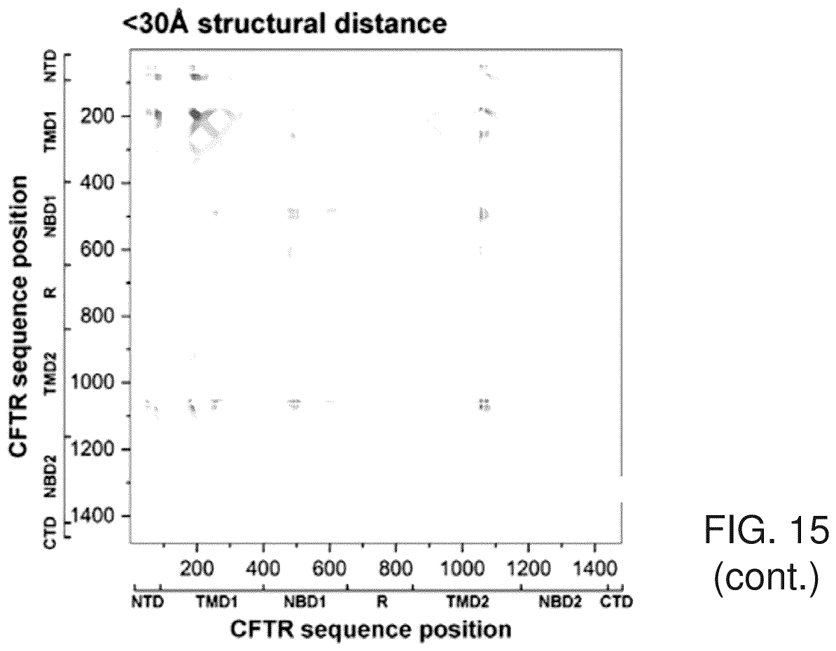

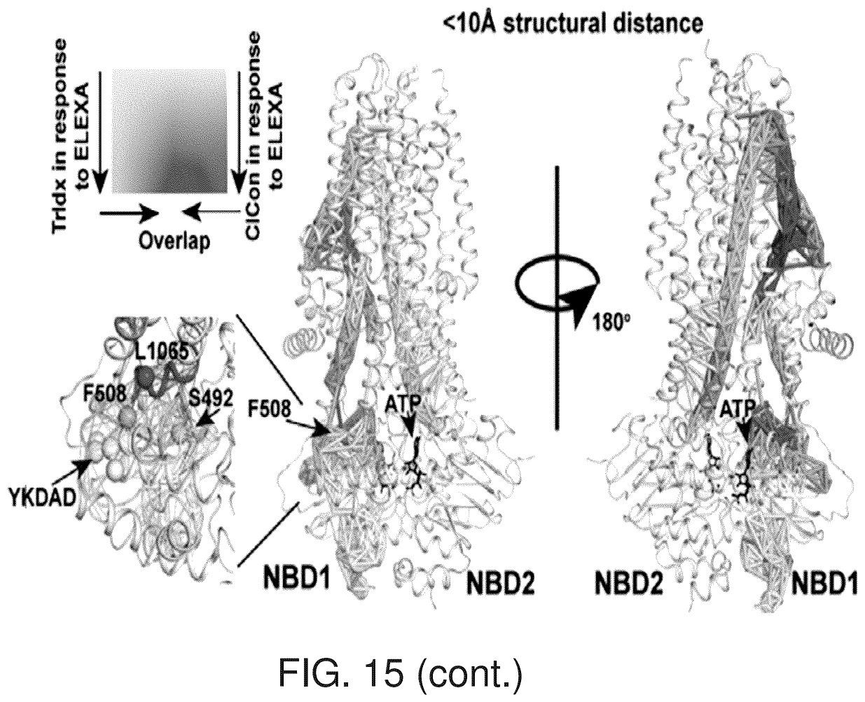

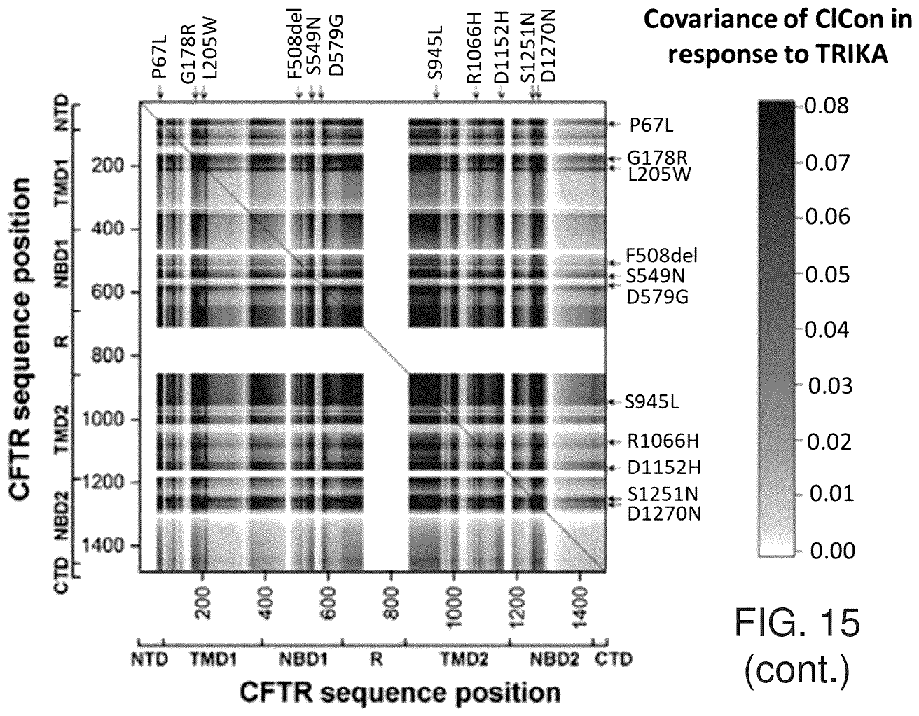

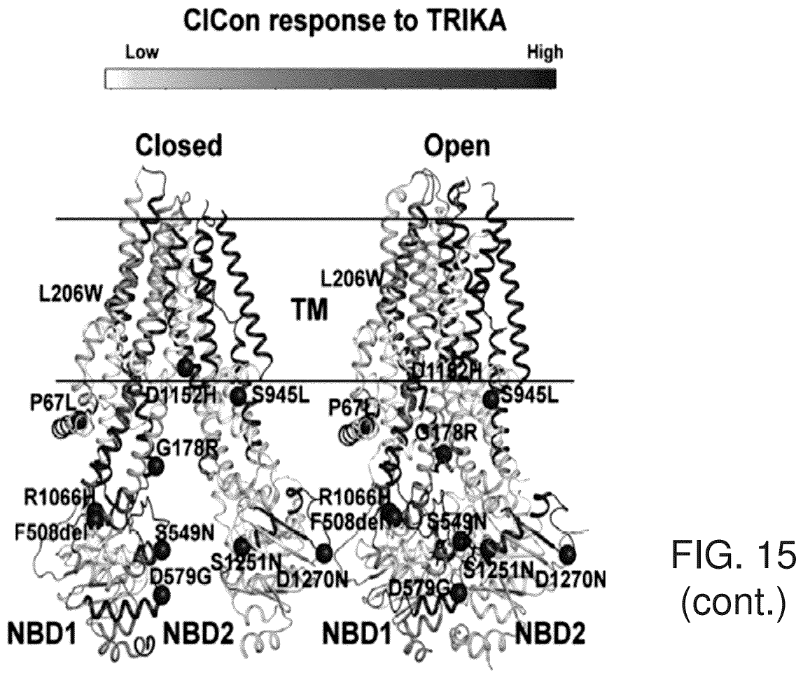

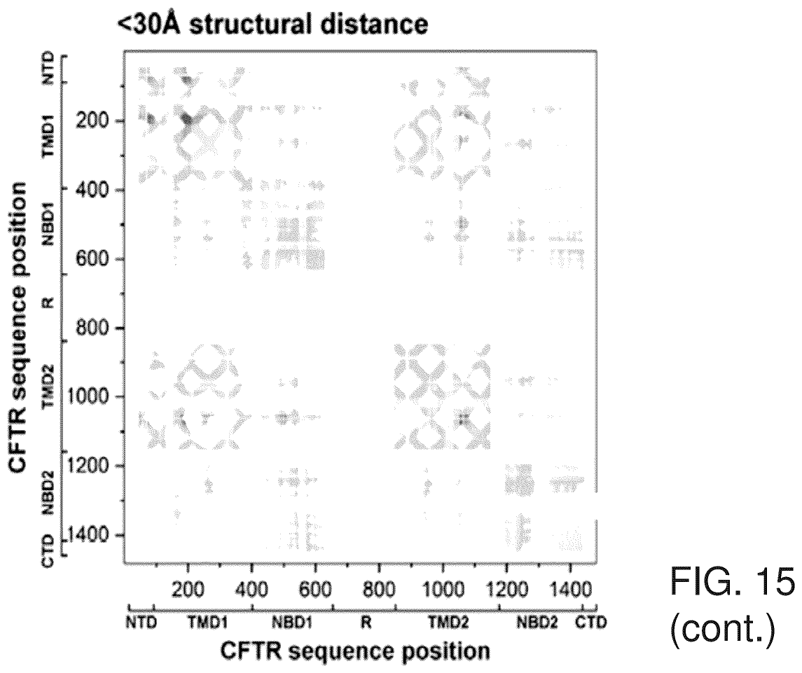

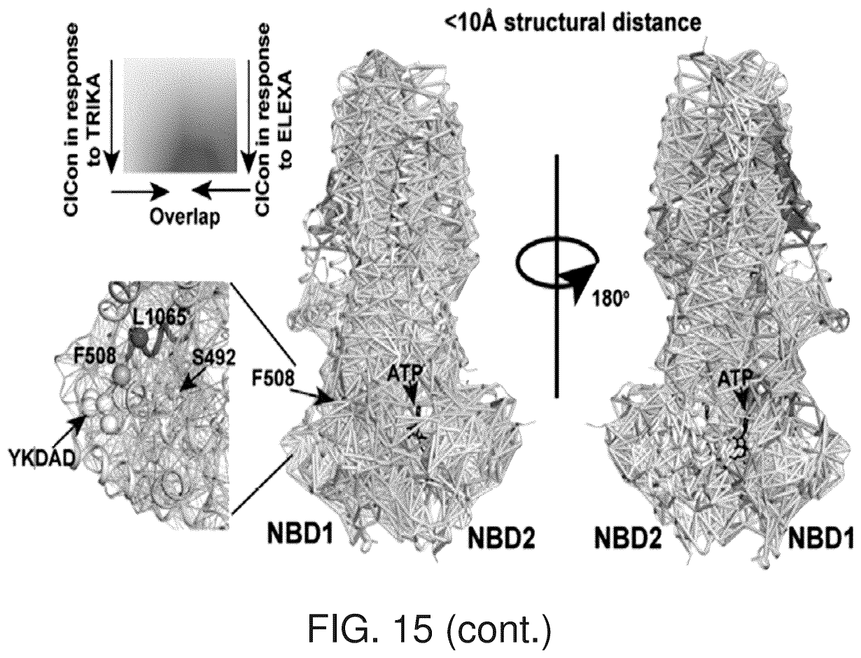

To assess how TrIdx is coupled to CICon at the cell surface in response to ELEXA or TRIKA, we assessed the steady-state CICon at the cell surface of WT and F508del. The result reveals that ELEXA or TRIKA treatment increases CICon function for F508del to ˜20% and ˜50% of WT, respectively. It suggests that TRIKA does not restore WT CFTR activity for F508del, consistent with ˜60% of WT short-circuit current measured in human nasal epithelia with CFTRF^{508del/F508del} and only ˜10-15% FEV1 improvement found in the clinic. To understand the impact of ELEXA and TRIKA on the SCV relationships spanning the entire CFTR polypeptide, we measured the CICon responses for 64 CF variants. We first generated an ELEXA/CICon-VarC map and the corresponding ELEXA/CICon-functional-response-structure (FIG. 15, upper left). The CICon sensitive sequence regions responding to ELEXA were similar to that for TrIdx, but with weaker covariance signals. Consistent with this observation, the overlapped MDC maps and MDC structures show that the TrIdx correction region (FIG. 15 upper middle MDC map, yellow and red) includes most of the CICon responsive regions (FIG. 15 upper right, red) with regions where the TrIdx correction is much higher than CICon correction (FIG. 15, upper right, yellow). These results indicate that there are additional properties of the fold dynamics found at the cell surface that are required for optimal channel gating that are not responsive to ELEXA corrected CFTR.

To understand the mechanisms driving CICon correction by TRIKA, we generated pairwise-residue-based TRIKA/CICon-VarC map and its corresponding functional TRIKA/CICon-functional-response-structure (FIG. 15, lower left), revealing a strikingly different covariance pattern from that observed for ELEXA alone. In contrast to the limited number regions impacted by ELEXA alone, most of the residues in the TRIKA/CICon-functional-response-structure have improved activity in the presence TRIKA—a result consistent with its improved impact in the clinic on CF pathophysiology. Overlap of the MDC maps and MDC structures describing the impact of TRIKA and ELEXA on CICon show the strong impact of the TRIKA on improving CICon compared to ELEXA (FIG. 9H, right panel, yellow). However, the YKDAD di-acidic code remains unresponsive, demonstrating the importance of correcting information flow defined by the genome in controlling the protein fold to achieve maximal function.

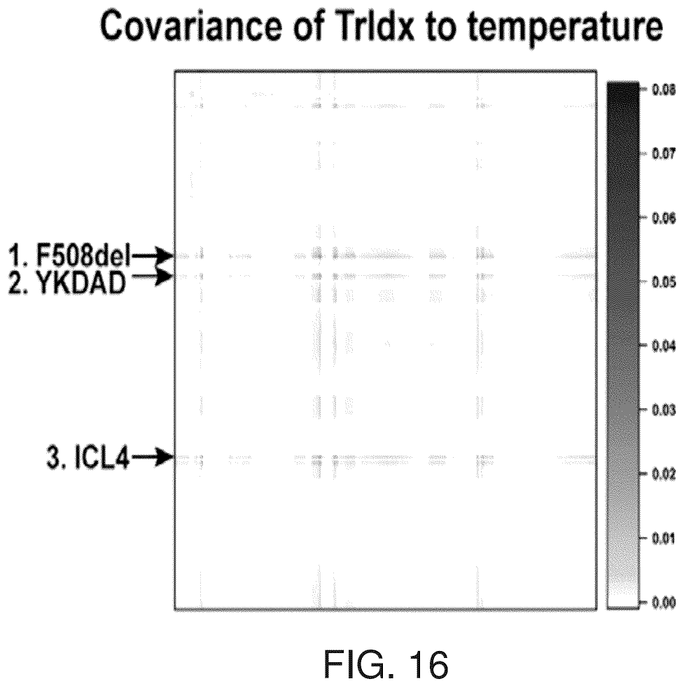

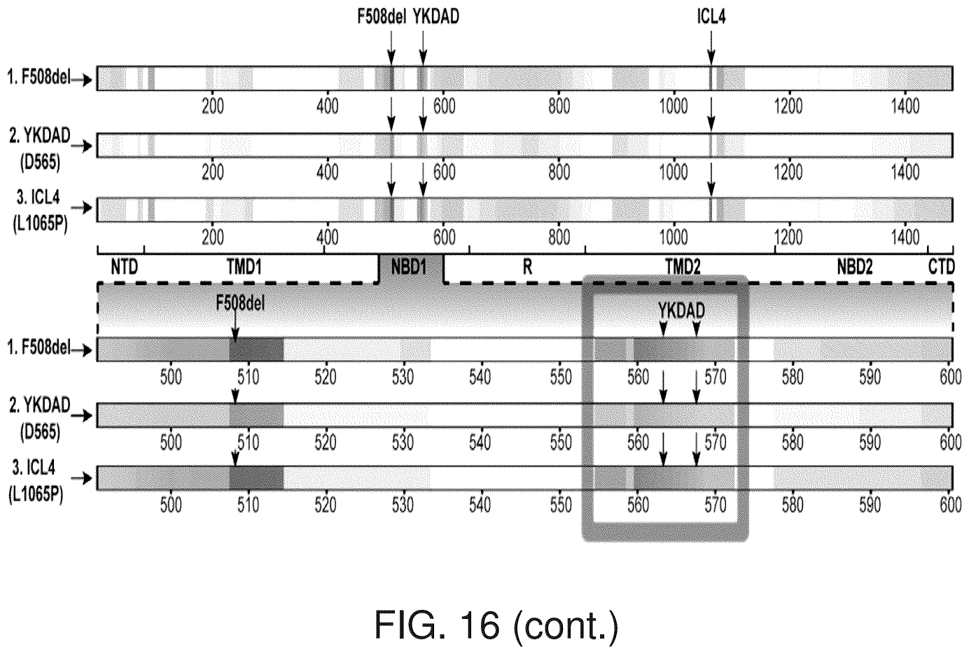

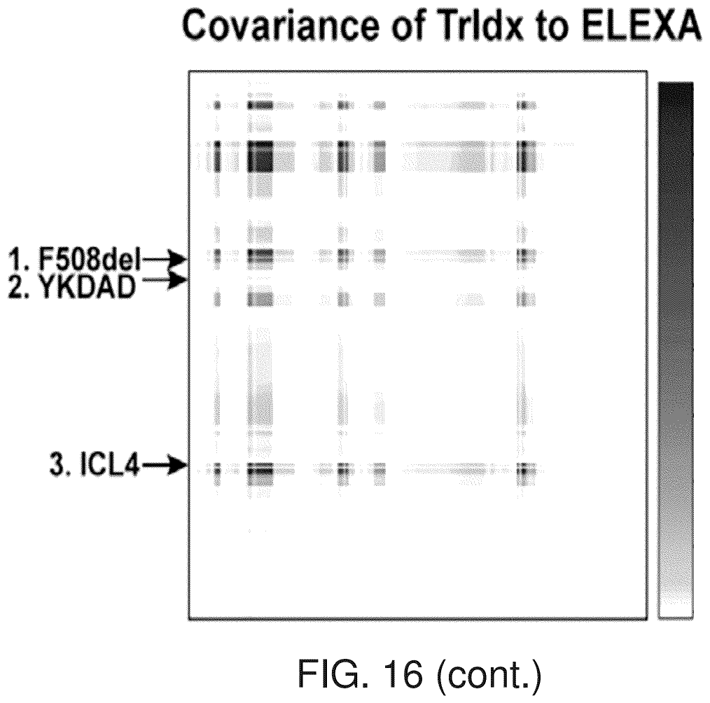

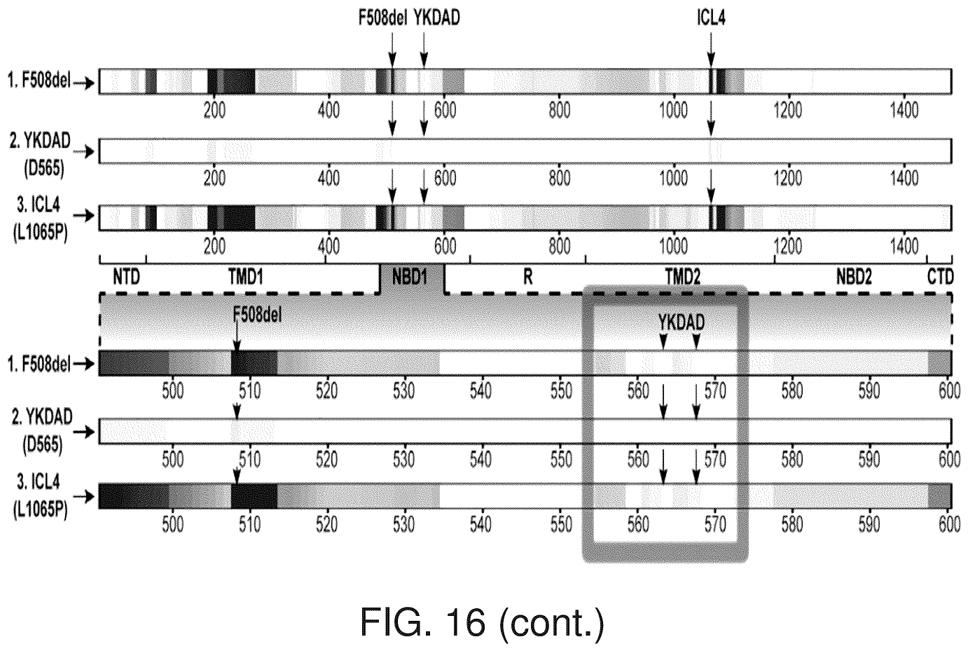

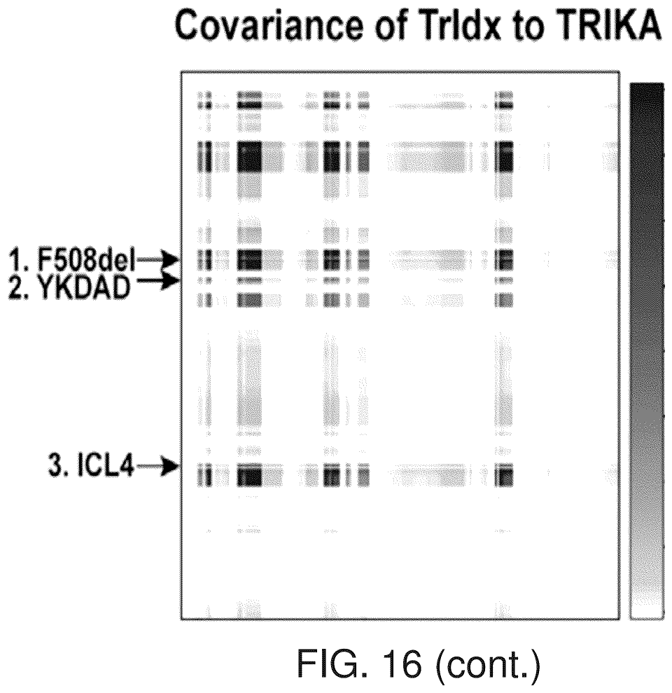

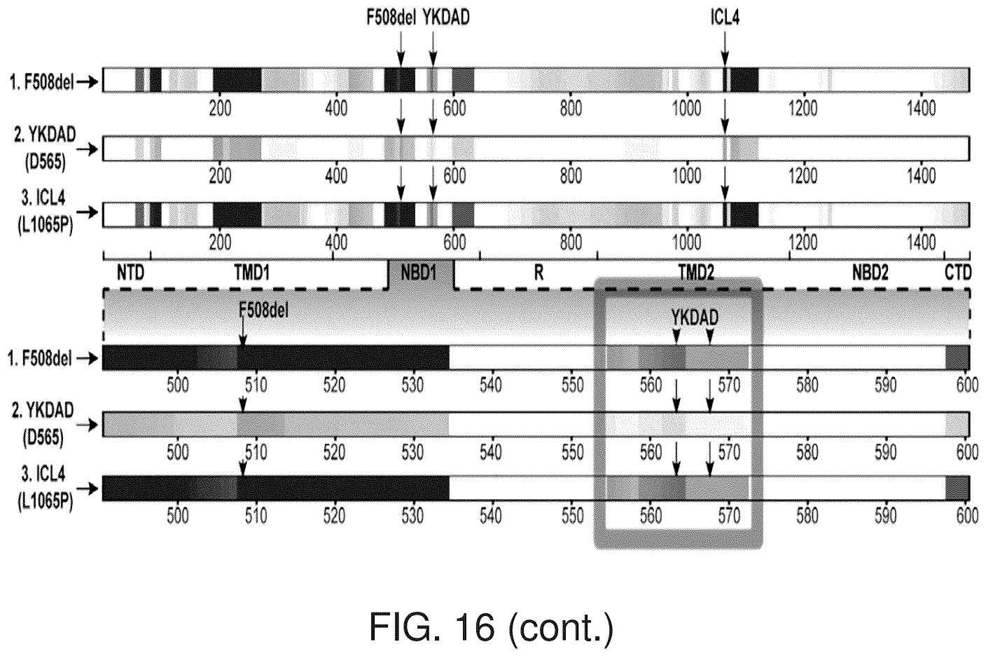

To provide a combined pairwise-residue description of the SCV relationships required to completely restore CFTR ER trafficking and cell surface CICon we focused on a triangulation approach to decipher more precisely the residue matrix contributing to disease. We extracted from each of the VarC plots all covariance values found in the polypeptide sequence responding to 3 key features of the fold namely F508 (FIG. 16, covariance-profile 1), D565 in the di-acidic code (563-YKDAD-567) (FIG. 16, covariance-profile 2) and L1065P in the ICL4 region (FIG. 16, covariance-profile 3). They locate in the top 3 responding regions to environmental modification by temperature shift, suggesting that these three structural regions (F508, ICL4 and YKDAD) cooperate with each other to form an SCV based energetically tuned functional core in CFTR fold design. Interestingly, while ELEXA dramatically increases the covariance of F508del and ICL4 to multiple non-temperature responsive residues including several sequence regions in TMD1 and enhances strong pairwise-residue covariance relationships between F508del and ICL4 (FIG. 16B, covariance-profiles 1, 3), ELEXA shows almost no impact on the pairwise-residue covariance relationships to the YKDAD di-acidic code contributing to either ER export (FIG. 16B; covariance-profile 2; inset zoom, covariance-profile 2, gray box) or CICon (covariance-profile 2). However, when ELEXA is examined in the context TRIKA mix, we can now detect combined pairwise-residue covariance interactions with the energetically tuned functional core containing the YKDAD motif (FIG. 16C, YKDAD, inset covariance-profile 2, gray box), but the signals are considerably lower than that observed for F508del and ICL4 (FIG. 16C, covariance-profile 1, 3). Furthermore, the predicted covariance response in or near the YKDAD energetic core for D565 is modest and significantly lower than that in temperature response (FIG. 16C, YKDAD, inset zoom covariance-profile 2, gray box vs FIG. 16A, YKDAD, inset zoom covariance-profile 2, gray box), suggesting a limited impact of TRIKA on the YKDAD associated SCV relationships contributing to trafficking to the cell surface.

Consistent with the VarC prediction, most of the variants surrounding YKDAD, for example L558S, A559T, R560T and Y569D, show little correction in response to TRIKA. One exception is R560K as this variant retains the positive charge to maintain the cation-IT interaction with F508 whereas the R560T variant with an uncharged side chain has limited response to TRIKA. However, R560T has a significant response to temperature shift emphasizing the importance of the core energetic features required for presentation of the YKDAD di-acidic code to the COPII vesicle budding machinery required for ER export. The results suggest that TRIKA operates mechanistically in a combined pairwise-residue network distinct from the temperature shift response, an SCV principled correction event through triangulation that will be critical for complete restoration of function of CFTR function at the cell surface.

To test whether we can add value to the pairwise-residue networks targeted by TRIKA to improve TRIKA benefit for CF by restoring the lost energetics of the fold in response to F508del affecting the function of di-acidic exit code, we measured the impact of ELEXA and TRIKA impact on F508del at 27° C. Here, we observe a dramatic improvement in the level of post-ER band C, total CFTR, and trafficking efficiency (band C relative to total) when compared to that at 37° C. These results demonstrate that the combined pair-wise residue relationships captured by triangulation of variation causing disease in the population using GPR-ML based VarC reveals the functional centerpiece of the CFTR fold, explaining the fundamental combined physical-chemical foundations responsible for CF.

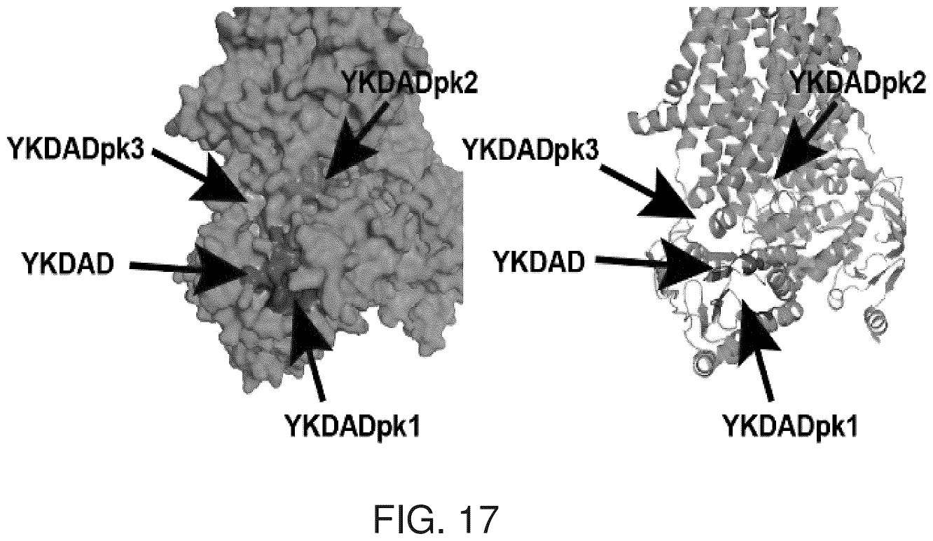

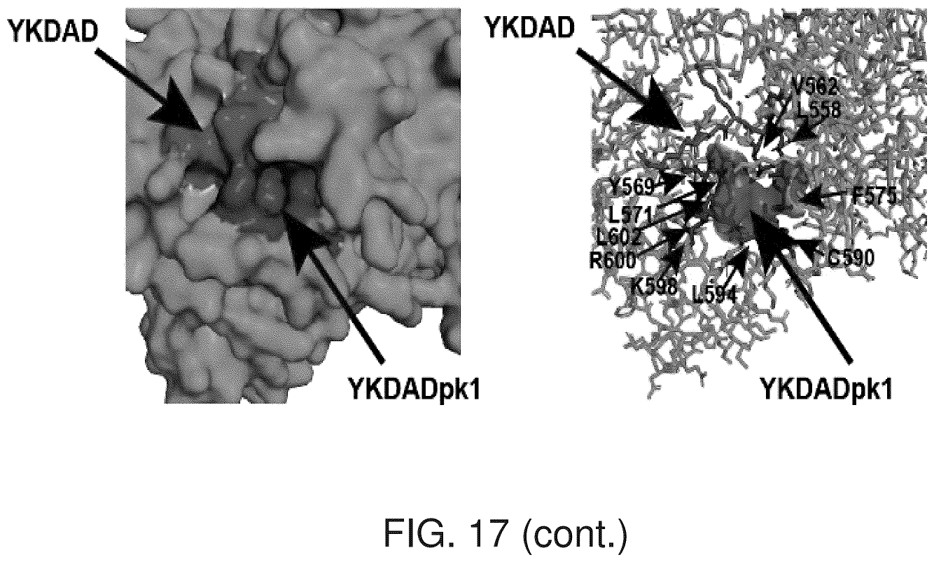

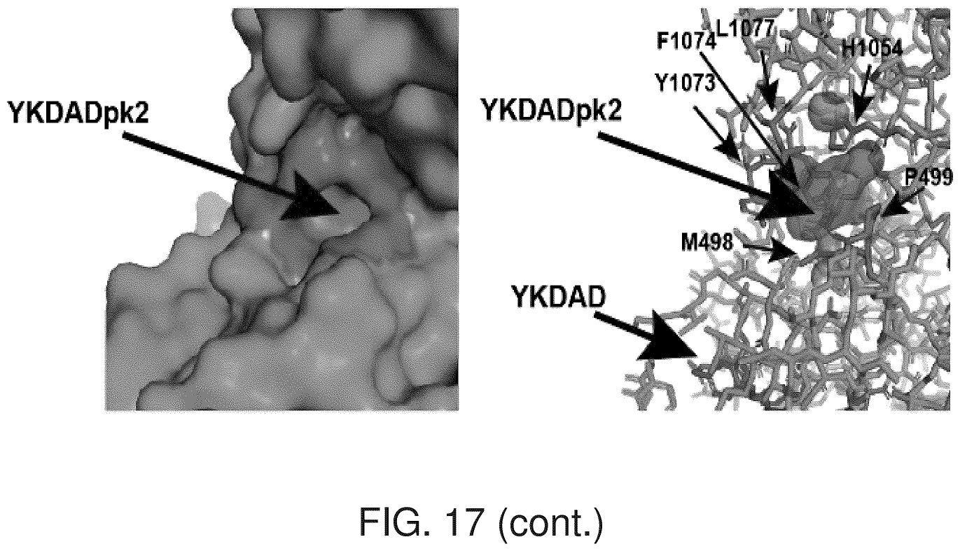

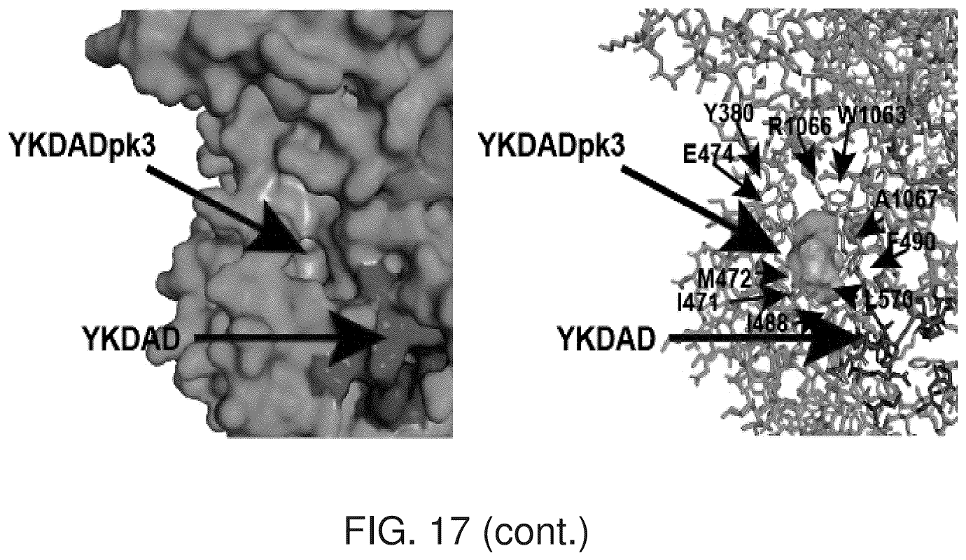

Referring now to FIG. 17, using GPR-ML, we showed that YKDAD motif in CFTR cannot be corrected by current FDA therapies. To search for chemical compounds in-silico that can stabilize the YKDAD motif, we performed an SCVT tool/method based in-silico computational screen to find the likely ligand-binding pockets for YKDAD stabilizer(s). We first use the program P2Rank/PrankWeb to predict all the possible ligand binding pockets in the closed conformation of CFTR structure (PDB:5UAK). Among all the predicted pockets, there are two predicted ligand-binding pockets nearby the YKDAD motif (FIG. 3A; YKDADpk1 and YKDADpk2). We also identified a ligand-binding pocket on the other side of YKDAD relative to the YKDADpk1 to set a triangulation platform using multiple residues for rescuing the YKDAD motif (FIG. 3A; YKDADpk3). YKDADpk1 (FIG. 3B) is formed by residues L558, V562, Y569, L571, F575, C590, L594, K598, R600 and L602 that are adjacent to YKDAD (residue 563-567) motif. The residues in this pocket do not respond to either LUMA or ELEXA (FIG. 2A). SCVT tool/method based in silico screening predicts that a small molecule binding to this site may stabilize YKDAD motif. YKDADpk2 (FIG. 3C) is formed by residues M498, P499 in nucleotide-binding domain 1 (NBD1) where the most common CF variant F508del is located, as well as residues H1054, Y1073, F1074 and L1077 in the intracellular loop 4 (ICL4) in the transmembrane domain 2 (TMD2). The inter-domain interactions between the ICL4 and NBD1 is crucial for CFTR folding. SCVT tool/method based in silico screening predicts that a small molecule that binds YKDADpk2 bridging ICL4 and NBD1 will likely improve the folding and trafficking of F508del. YKDADpk3 (FIG. 3D) is formed by residue Y380 in transmembrane domain 1 (TMD1), 1471, M472, E474, 1488, L570, F490 in NBD1, and W1063, R1066 and A1067 in ICL4. This pocket involves many inter-domain interactions (including the critical NBD1-ICL4 interactions) and very close to the YKDAD motif. SCVT tool/method based in silico screening predicts a small molecule binding to this pocket may stabilize CFTR fold for ER export. The example of CFTR using SCVT based methodology revealing the three defined ligand-binding pockets illustrates the general utility of the SCVT tool to screen databases including ZINC15 that has over 230 million purchasable compounds by using Maestro software and Autodock, or the Enamine 1.1 billion compound collection (among others) to enable a high precision drug discovery effort linked to sequence based functional impacts of variants found in the population for any disease.

For CF, the functional energetic core harboring the YKDAD motif revealed by low temperature shift was found to be chemically inaccessible to modulation by ELEXA alone and only partially affected by TRIKA mix. VarC revealed it is one part of a triad of domain interactions including YKDAD, F508 and ICL4 with only the latter two regions being partially impacted by TRIKA.

The therapeutic nonresponsive YKDAD (residues 563-567) motif region is linked to variants found in CF population including the amino-terminal flanking R560T that disrupts the cation-TT interaction with F508, L558S facing the opposite site side of the R560-F508 linkage, and the C-terminal flanking Y569D variant. As each of these variants and other SCV predicted residues are responsive to temperature shift, our results raise the possibility that we can now use a SCV principled approach to triangulate a solution to the apparent unresponsive part of the functional energetic core responsible for clinical disease. For example, a small molecule screen against flanking variants indicated above is anticipated to yield a more complete solution to the YKDAD folding and trafficking problem. Such a triangulation-based screening effort could provide the missing link in generation of a remodeling event(s) that either in the absence or, more likely, presence of TRIKA corrects the missing feature of the fold that will be necessary to ‘cure’ disease by restoration to near WT function.

Several important conclusions result from the above description of the novel methods set forth herein. First, by triangulation of the SCV relationships (SCVT) through GPR-ML, we can use sparse variant information to identify the key features of the entire protein fold at atomic resolution that cannot be corrected by current chemical therapies. For example, we show for the first time that the YKDAD motif cannot be corrected by TRIKA, which presents a limitation of TRIKA in the clinic. This is a generally applicable principle for any biological/clinical activity associated with treatment of human disease with small molecule therapeutics.

Second, the comparison between the SCVT tool/method-based responses to chemical treatment and thermodynamic modulation provide a novel platform to pinpoint the target for the development of pharmacological chaperone.

Third, using the SCVT tool/method, we provide a novel strategy to guide experimental HTS/HCIS/in silico computational screening by triangulating fold management in response to variants triggering human genetic disease. For example, we use the surrounding variants contributing to stability of the YKDAD motif that can be thermodynamically rescued to screen for novel pharmacological chaperones that improve YKDAD motif stability to restore critical lost function to CFTR.

Fourth, SCVT based understanding of the responses of a sparse collection of variants found in the population predicts the response (function) of every residue in the entire structure to existing drugs which, when combined with standard structural modeling technologies, can identify the potential binding pockets for precision structure-based drug design.

Fifth, we provide a novel molecular platform for in-silico drug screening using the SCVT tool/method. Since we can functionally define residue-by-residue and residue-to-residue drug responses for the entire protein structure through GPR-ML, we can triangulate drug binding pockets or potential new ligand-binding pockets for in-silico screening in the search for new small molecules that stabilize the fold for function, the example being the ligand-binding pockets found to stabilize YKDAD motif in CFTR through SCVT.

Sixth, since we can map the Achilles heel in the structure that cannot be corrected by existing drugs through application of SCTV method using GPR-ML, we can predict potential ligand-binding pockets in a SCV-based triangulation platform to discover small molecules with new mechanisms of action understanding prior to extensive clinical trials. Thus, unlike normal high-throughput screening which simply goes for a “corrector” effect based on an assay, VarC visualizations and analysis such as described herein can elucidate what needs to be fixed to achieve functional correction. This enables application of additional computational approaches to define with precision on the correct compound design to, for example, modulate the fold to fix the problem.

### General Interpretive Principles

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure. For example, a system or an apparatus may be implemented, or a method may be practiced using any one or more of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

With respect to the use of plural vs. singular terms herein, those having skill in the art can translate from the plural to the singular and/or from the singular to the plural as is appropriate to the context and/or application. The various singular/plural permutations may be expressly set forth herein for sake of clarity.

When describing an absolute value of a characteristic or property of a thing or act described herein, the terms “substantial,” “substantially,” “essentially,” “approximately,” and/or other terms or phrases of degree may be used without the specific recitation of a numerical range. When applied to a characteristic or property of a thing or act described herein, these terms refer to a range of the characteristic or property that is consistent with providing a desired function associated with that characteristic or property.

In those cases where a single numerical value is given for a characteristic or property, it is intended to be interpreted as at least covering deviations of that value within one significant digit of the numerical value given.

If a numerical value or range of numerical values is provided to define a characteristic or property of a thing or act described herein, whether or not the value or range is qualified with a term of degree, a specific method of measuring the characteristic or property may be defined herein as well. In the event no specific method of measuring the characteristic or property is defined herein, and there are different generally accepted methods of measurement for the characteristic or property, then the measurement method should be interpreted as the method of measurement that would most likely be adopted by one of ordinary skill in the art given the description and context of the characteristic or property. In the further event there is more than one method of measurement that is equally likely to be adopted by one of ordinary skill in the art to measure the characteristic or property, the value or range of values should be interpreted as being met regardless of which method of measurement is chosen.

It will be understood by those within the art that terms used herein, and especially in the appended claims (e.g., bodies of the appended claims) are intended as “open” terms unless specifically indicated otherwise (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

In those instances where a convention analogous to “at least one of A, B, and C” is used, such a construction would include systems that have A alone, B alone, C alone, A and B together without C, A and C together without B, B and C together without A, as well as A, B, and C together. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include A without B, B without A, as well as A and B together.”

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A method of estimating clinical, biological and/or chemical properties of protein variants, the method comprising:
estimating one or more clinical, biological, and/or chemical properties in the presence and absence of one or more therapeutic and/or environmental conditions for amino acid residue variation at all or substantially all amino acids of the amino acid sequence of the protein;
estimating pairwise covariance of the estimated clinical, biological, and/or chemical properties in the presence and absence of at least one therapeutic or environmental condition for all or substantially all amino acid pairs of the protein;
estimating a 3D distance between all or substantially all amino acid pairs of the protein;
generating a visualization comprising a combination of the estimated pairwise covariance and the estimated 3D distance.

2. The method of claim 1, wherein the estimating one or more clinical, biological, and/or chemical properties in the presence and absence of one or more therapeutic and/or environmental conditions for amino acid residue variation at all or substantially all amino acids of the amino acid sequence of the protein comprises receiving information regarding clinical, biological, and/or chemical properties in the presence and absence of one or more therapeutic and/or environmental conditions for a plurality of known naturally occurring amino acid residue variants of the protein.

3. The method of claim 1, wherein generating the visualization comprises generating and displaying a 2-dimensional map of pairwise covariance estimates filtered by a 3D distance threshold.

4. The method of claim 3, wherein the 3D distance threshold is between 10 angstroms and 50 angstroms.

5. The method of claim 4, wherein the 3D distance threshold is about 30 angstroms.

6. The method of claim 1, wherein the 3D distance is measured from the alpha carbon of the amino acids.

7. The method of claim 1, wherein the estimating a 3D distance is based at least in part on an experimentally or computationally determined 3D protein structure.

8. The method of claim 1, wherein the visualization comprises a two-dimensional matrix with amino acid position in the sequence on both the x-axis and the y-axis.

9. The method of claim 1, wherein the degree of covariance for a given pair of amino acid residues is indicated by a color and/or a shade of a color.

10. A method of estimating clinical, biological and/or chemical properties of protein variants, the method comprising:
estimating one or more clinical, biological, and/or chemical properties in the presence and absence of a first therapeutic and/or environmental condition for amino acid residue variation at all or substantially all amino acids of the amino acid sequence of the protein;
estimating one or more clinical, biological, and/or chemical properties in the presence and absence of a second therapeutic and/or environmental condition for amino acid residue variation at all or substantially all amino acids of the amino acid sequence of the protein;
estimating pairwise covariance of the estimated clinical, biological, and/or chemical properties in the presence and absence of the first therapeutic or environmental condition for all or substantially all amino acid pairs of the protein;
estimating pairwise covariance of the estimated clinical, biological, and/or chemical properties in the presence and absence of the second therapeutic or environmental condition for all or substantially all amino acid pairs of the protein;
generating a visualization comprising a combination of the estimated pairwise covariance of the clinical, biological, and/or chemical properties in the presence and absence of the first therapeutic or environmental condition for all or substantially all amino acid pairs of the protein and the estimated clinical, biological, and/or chemical properties in the presence and absence of the second therapeutic or environmental condition for all or substantially all amino acid pairs of the protein.

11. The method of claim 10, wherein the first therapeutic and/or environmental condition comprises exposure to a first chemical compound.

12. The method of claim 11 wherein the second therapeutic and/or environmental condition comprises exposure to a second chemical compound.

13. The method of claim 11, wherein the second therapeutic and/or environmental condition comprises an environmental condition.

14. The method of claim 13, wherein the environmental condition comprises a temperature shift from normal body temperature.

15. The method of claim 10, wherein the visualization comprises a two-dimensional matrix with amino acid position in the sequence on both the x-axis and the y-axis.

16. The method of claim 10, wherein the degree of covariance for a given pair of amino acid residues is indicated by a color and/or a shade of a color.
